(19) United States
(12) Patent Application Publication
(10) Pub. No.: US 2021/0322737 A1
(43) Pub. Date: Oct. 21, 2021

(54) COMPOSITIONS AND METHODS FOR REDUCING TRAUMATIC EDEMA FROM SEVERE SPINAL CORD INJURY

(71) Applicant: THE REGENTS OF THE UNIVERSITY OF CALIFORNIA, Oakland, CA (US)

(72) Inventors: Victor G.J. Rodgers, San Bernardino, CA (US); Devin K. Binder, Irvine, CA (US); Christopher Hale, Palm Desert, CA (US); Jennifer Yonan, Irvine, CA (US)

(21) Appl. No.: 17/229,587

(22) Filed: Apr. 13, 2021

Related U.S. Application Data
(60) Provisional application No. 63/009,949, filed on Apr. 14, 2020.

(51) Int. Cl. A61M 27/00

(52) U.S. Cl. CPC A61M 27/006; A61M 27/008; A61M 2205/3303; A61M 2205/3368; A61M 2205/3327; A61M 2210/1003

(57) ABSTRACT

A continuous-flow system for the treatment of edema in an injured central nervous system (CNS) tissue, including: a reversibly implantable device having: an inflow pathway, an outflow pathway, and a fluid flow pathway connecting the first outlet of the inflow pathway and the second inlet of the outflow pathway, wherein the fluid flow pathway includes a semi-permeable membrane; a first reservoir; a fluid-driving apparatus; a second reservoir; and a plurality of fluid flow conduits that fluidically connect the first reservoir, the fluid-driving apparatus, the second reservoir, and the reversibly implantable device; wherein the reversibly implantable device is configured to allow direct contact between the semi-permeable membrane and at least a portion of the injured CNS tissue; wherein the system is configured to contain a solution that pass through the fluid flow pathway and induces osmotic flow of water from the injured CNS tissue across the semipermeable membrane and into the solution, thereby decreasing swelling of the tissue. Also disclosed are related methods for removing water from a traumatically injured central nervous system (CNS) tissue in a subject.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The invention generally relates to medicine and medical devices. The invention provides compositions and methods for the treatment and/or reversal of an edema, e.g., including a central nervous system (CNS) edema, e.g., a spinal edema, a spinal injury or insult.

### Description of the Related Art

It is estimated that between ¼ to ½ million people will endure a spinal cord injury (SCI) each year, world-wide (World Health Organization, and International Spinal Cord Society (2013). International Perspectives on Spinal Cord Injury. Geneva: World Health Organization). SCI causes long-lasting and often irreversible changes in motor, sensory and autonomic function, leading to reduced quality of life and increased morbidity rates in those affected (Barker, R. N., Kendall, M. D., Amsters, D. I., Pershouse, K. J., Haines, T. P., and Kuipers, P. (2009). The relationship between quality of life and disability across the lifespan for people with spinal cord injury. Spinal Cord 47, 149-155; Hagen, E. M., Lie, S. A., Rekand, T., Gilhus, N. E., and Gronning, M. (2010). Mortality after traumatic spinal cord injury: 50 years of follow-up. J. Neurol. Neurosurg. Psychiatry 81, 368-373). SCI is characterized by the initial injury due to trauma, and by secondary cellular events that result in a further tissue damage. The period of secondary injury is accompanied by breakdown of the blood-spinal cord barrier (BSCB), hemorrhage, edema, ischemia, inflammation, and tissue necrosis at and around the injury site (Whetstone, W. D., Hsu, J. Y. C., Eisenberg, M., Werb, Z., and Noble-Haeusslein, L. J. (2003). Blood-spinal cord barrier after spinal cord injury: relation to revascularization and wound healing. J. Neurosci. Res. 74, 227-239; Norenberg, M. D., Smith, J., and Marcillo, A. (2004). The pathology of human spinal cord injury: defining the problems. J. Neurotrauma 21, 429-440; Borgens, R. B., and Liu-Snyder, P. (2012). Understanding secondary injury. Q. Rev. Biol. 87, 89-127). Edema levels (cytotoxic, vasogenic, or both) increase within the first few hours after injury (Leypold, B. G., Flanders, A. E., and Burns, A. S. (2008). The early evolution of spinal cord lesions on MR imaging following traumatic spinal cord injury. AJNR Am. J. Neuroradiol. 29, 1012-1016) and are correlated with poorer neurological outcome and reduced independence (Flanders, A. E., Schaefer, D. M., Doan, H. T., Mishkin, M. M., Gonzalez, C. F., and Northrup, B. E. (1990). Acute cervical spine trauma—correlation of MR imaging findings with degree of neurologic deficit. Radiology 177, 25-33, Flanders, A. E., Spettell, C. M., Tartaglino, L. M., Friedman, D. P., and Herbison, G. J. (1996). Forecasting motor recovery after cervical spinal cord injury: value of MR imaging. Radiology 201, 649-655, Flanders, A. E., Spettell, C. M., Friedman, D. P., Marino, R. J., and Herbison, G. J. (1999). The relationship between the functional abilities of patients with cervical spinal cord injury and the severity of damage revealed by MR imaging. Am. J. Neuroradiol. 20, 926-934). Larger increases in edema levels are observed in individuals with more severe injuries and reduced recovery following injury (Shepard, M. J., and Bracken, M. B. (1999). Magnetic resonance imaging and neurological recovery in acute spinal cord injury: observations from the National Acute Spinal Cord Injury Study 3. Spinal Cord 37, 833-837; Boldin, C., Raith, J., Fankhauser, F., Haunschmid, C., Schwantzer, G., and Schweighofer, F. (2006). Predicting neurologic recovery in cervical spinal cord injury with postoperative MR imaging. Spine 31, 554-559; Bozzo, A., Marcoux, J., Radhakrishna, M., Pelletier, J., and Goulet, B. (2011). The role of magnetic resonance imaging in the management of acute spinal cord injury. J. Neurotrauma 28, 1401-1411). Spinal cord edema is also associated with both cord swelling and compression (Miyanji, F., Furlan, J. C., Aarabi, B., Arnold, P. M., and Fehlings, M. G. (2007). Acute cervical traumatic spinal cord injury: MR imaging findings correlated with neurologic outcome-prospective study with 100 consecutive patients. Radiology 243, 820-827) which has been correlated with worse neurological outcome (Werndle, M. C., Saadoun, S., Phang, I., Czosnyka, M., Varsos, G. V., Czosnyka, Z. H., et al. (2014). Monitoring of spinal cord perfusion pressure in acute spinal cord injury: initial findings of the injured spinal cord pressure evaluation study*. Crit. Care Med. 42, 646-655; Papadopoulos, M. C. (2015). Intrathecal pressure after spinal cord injury. Neurosurgery 77:E500; Phang, I., and Papadopoulos, M. C. (2015). Intraspinal pressure monitoring in a patient with spinal cord injury reveals different intradural compartments: injured spinal cord pressure evaluation (ISCoPE) Study. Neurocrit. Care 23, 414-418). Unfortunately, surgical decompression and stabilization do not reduce edema or minimize the resulting ischemia-induced necrosis (Saadoun, S., and Papadopoulos, M. C. (2010). Aquaporin-4 in brain and spinal cord oedema. Neuroscience 168, 1036-1046). In addition, its use in various SCI models along with its window of effectiveness remain controversial (Fehlings, M. G., and Perrin, R. G. (2006). The timing of surgical intervention in the treatment of spinal cord injury: a systematic review of recent clinical evidence. Spine 31(11 Suppl.), S28-S35). Further, the use of methylprednisolone (MP) to reduce edema and ischemia is waning due to controversy over its beneficial and harmful effects (Braughler, J. M., and Hall, E. D. (1982). Correlation of methylprednisolone levels in cat spinal cord with its effects on (Na++K+)-ATPase, lipid peroxidation, and alpha motor neuron function. J. Neurosurg. 56, 838-844, 1982; Hall, E. D., Wolf, D. L., and Braughler, J. M. (1984). Effects of a single large dose of methylprednisolone sodium succinate on experimental posttraumatic spinal cord ischemia. Dose-response and time-action analysis. J. Neurosurg. 61, 124-130; Cayli, S. R., Kocak, A., Yilmaz, U., Tekiner, A., Erbil, M., Ozturk, C., et al. (2004). Effect of combined treatment with melatonin and methylprednisolone on neurological recovery after experimental spinal cord injury. Eur. Spine J. 13, 724-732; Rozet, I. (2008). Methylprednisolone in acute spinal cord injury: is there any other ethical choice? J. Neurosurg. Anesthesiol. 20, 137-139). Still other research has looked into the beneficial effects of hypertonic saline (Nout, Y. S., Mihai, G., Tovar, C. A., Schmalbrock, P., Bresnahan, J. C., and Beattie, M. S. (2009). Hypertonic saline attenuates cord swelling and edema in experimental spinal cord injury: a study utilizing magnetic resonance imaging. Crit. Care Med. 37, 2160-2166) and the use of a mechanical tissue resuscitation device (Zheng, Z. L., Morykwas, M. J., Tatter, S., Gordon, S., McGee, M., Green, H., et al. (2015). Ameliorating spinal cord injury in an animal model with mechanical tissue resuscitation. Neurosurgery [Epub ahead of print]) to minimize histological damage.

Recently, a series of significant clinical data in the Injured Spinal Cord Pressure Evaluation (ISCoPE) study has emerged indicating the importance of intraspinal pressure (ISP) at the injury site in outcome after SCI (Werndle, M. C., Saadoun, S., Phang, I., Czosnyka, M., Varsos, G. V., Czosnyka, Z. H., et al. (2014). Monitoring of spinal cord perfusion pressure in acute spinal cord injury: initial findings of the injured spinal cord pressure evaluation study. Crit. Care Med. 42, 646-655; Papadopoulos, M. C. (2015). Intrathecal pressure after spinal cord injury. Neurosurgery 77:E500; Phang and Papadopoulos, 2015; Phang et al., 2015; Varsos et al., 2015). These studies showed that: (i) ISP after SCI is elevated as the swollen cord is compressed against the dura; (ii) spinal cord perfusion pressure (SCPP) decreases at the site of injury and impacts outcome; and (iii) laminectomy with expansion duraplasty compared to decompressive laminectomy alone reduces ISP, increases SCPP, and leads to greater decompression of the injured cord (Phang, I., and Papadopoulos, M. C. (2015). Intraspinal pressure monitoring in a patient with spinal cord injury reveals different intradural compartments: injured spinal cord pressure evaluation (ISCoPE) Study. Neurocrit. Care 23, 414-418; Chen, S. L., Smielewski, P., Czosnyka, M., Papadopoulos, M. C., and Saadoun, S. (2017). Continuous monitoring and visualization of optimum spinal cord perfusion pressure in patients with acute cord injury. J. Neurotrauma 34, 2941-2949; Chen, S., Gallagher, M. J., Papadopoulos, M. C., and Saadoun, S. (2018). Non-linear dynamical analysis of intraspinal pressure signal predicts outcome after spinal cord injury. Front. Neurol. 9:493; Gallagher, M. J., Hogg, F. R. A., Zoumprouli, A., Papadopoulos, M. C., and Saadoun, S. (2019). Spinal cord blood flow in patients with acute spinal cord injuries. J. Neurotrauma 36, 919-929; Hogg, F. R. A., Gallagher, M. J., Chen, S., Zoumprouli, A., Papadopoulos, M. C., and Saadoun, S. (2019). Predictors of intraspinal pressure and optimal cord perfusion pressure after traumatic spinal cord injury. Neurocrit. Care 30, 421-428). These findings have also been corroborated in rodent and porcine models of SCI (Saadoun, S., Bell, B. A., Verkman, A. S., and Papadopoulos, M. C. (2008). Greatly improved neurological outcome after spinal cord compression injury in AQP4-deficient mice. Brain 131(Pt 4), 1087-1098; Leonard, A. V., Thornton, E., and Vink, R. (2015). The relative contribution of edema and hemorrhage to raised intrathecal pressure after traumatic spinal cord injury. J. Neurotrauma 32, 397-402; Khaing, Z. Z., Cates, L. N., Fischedick, A. E., McClintic, A. M., Mourad, P. D., and Hofstetter, C. P. (2017). Temporal and spatial evolution of raised intraspinal pressure after traumatic spinal cord injury. J. Neurotrauma 34, 645-651; Streijger, F., So, K., Manouchehri, N., Tigchelaar, S., Lee, J. H. T., Okon, E. B., et al. (2017). Changes in pressure, hemodynamics, and metabolism within the spinal cord during the first 7 days after injury using a porcine model. J. Neurotrauma 34, 3336-3350). These initial studies suggest that spinal cord parenchymal swelling due to edema accumulation continues to expand radially until the tissue reaches the dura and can no long swell outward, despite routine decompressive laminectomy. This leads to an inevitable localized pressure build-up that causes the subarachnoid space to collapse at the epicenter and significant constriction of flow within local blood vessels (Soubeyrand, M., Badner, A., Vawda, R., Chung, Y. S., and Fehlings, M. G. (2014a). Very high-resolution ultrasound imaging for real-time quantitative visualization of vascular disruption after spinal cord injury. J. Neurotrauma 31, 1767-1775; Khaing, Z. Z., Cates, L. N., DeWees, D. M., Hannah, A., Mourad, P., Bruce, M., et al. (2018). Contrast-enhanced ultrasound to visualize hemodynamic changes after rodent spinal cord injury. J. Neurosurg. Spine 29, 306-313; Saadoun, S., and Papadopoulos, M. C. (2020). Targeted perfusion therapy in spinal cord trauma. Neurotherapeutics 17, 511-521). The collapsed blood vessels are no longer able to supply nutrients to the surrounding tissue and this creates local ischemia, further worsening tissue secondary injury (Gallagher, M. J., Hogg, F. R. A., Zoumprouli, A., Papadopoulos, M. C., and Saadoun, S. (2019). Spinal cord blood flow in patients with acute spinal cord injuries. J. Neurotrauma 36, 919-929).

These key new clinical data and recent animal models indicate the importance of developing innovative treatments aimed at preventing or reversing spinal cord edema and subsequent swelling following injury. To date there is no widely accepted and effective treatment for edema following SCI. It is widely accepted, however, that early intervention may limit the amount of secondary damage. There is, therefore, a need for new methods to effectively ameliorate edema following SCI in order to minimize spinal cord compression, decrease ISP at the injury site, improve vascular perfusion (SCPP), and improve neurological outcome. In this work we develop our currently effective osmotic transport device (OTD) that has been shown to improve outcome in global and focal models of cerebral edema (McBride, D. W., Hsu, M. S., Rodgers, V. G. J., and Binder, D. K. (2012). Improved survival following cerebral edema using a novel hollow fiber-hydrogel device. J. Neurosurg. 116, 1389-1394, McBride, D. W., Szu, J. I., Hale, C., Hsu, M. S., Rodgers, V. G., and Binder, D. K. (2014). Reduction of cerebral edema after traumatic brain injury using an osmotic transport device. J. Neurotrauma 31, 1948-1954, McBride, D. W., Donovan, V., Hsu, M. S., Obenaus, A., Rodgers, V., and Binder, D. K. (2016). “Reduction of cerebral edema via an osmotic transport device improves functional outcome after traumatic brain injury in mice,” in Brain Edema XVI, eds R. Applegate, G. Chen, H. Feng, and J. Zhang (Berlin: Springer), 285-289) and apply it to SCI in a well-accepted rodent model of thoracic contusion SCI.

We have recently demonstrated that through establishing an external osmotic gradient, water can be removed from the brain in a controlled manner under normal and pathological brain swelling conditions. We found that the OTD reduced tissue water content and dramatically improved neurological outcome in an acute mouse models of cytotoxic edema and traumatic brain injury (TBI induced by controlled cortical impact, CCI) without causing histological damage (McBride, D. W., Hsu, M. S., Rodgers, V. G. J., and Binder, D. K. (2012). Improved survival following cerebral edema using a novel hollow fiber-hydrogel device. J. Neurosurg. 116, 1389-1394, McBride, D. W., Szu, J. I., Hale, C., Hsu, M. S., Rodgers, V. G., and Binder, D. K. (2014). Reduction of cerebral edema after traumatic brain injury using an osmotic transport device. J. Neurotrauma 31, 1948-1954, McBride, D. W., Donovan, V., Hsu, M. S., Obenaus, A., Rodgers, V., and Binder, D. K. (2016). “Reduction of cerebral edema via an osmotic transport device improves functional outcome after traumatic brain injury in mice,” in Brain Edema XVI, eds R. Applegate, G. Chen, H. Feng, and J. Zhang (Berlin: Springer), 285-289; and U.S. Pat. No. 10,420,918). These results established proof-of-principle for the concept of direct osmotherapy for treatment of CNS edema.

### SUMMARY OF THE INVENTION

We demonstrate that an osmotic transport device OTD, placed on the dura mater of the spinal cord at the site of injury, can withdraw fluid from the cord by permeation through the adjacent tissue, thereby reducing swelling and providing relief of vasculature compression. FIG. 1 provides a simplified model of the dynamics of tissue compartments in SCI and how the OTD ameliorates SCI.

In 4-hour, blunt trauma SCI studies with rats (OTD applied one hour after injury followed by 3 h of operation), we showed that our spinal cord OTD significantly reduces edema, as determined by tissue water content at the injury site. We describe the importance of this reduction and discuss how reduction of swelling may significantly open flow in the subarachnoid space and spinal cord tissue itself, potentially reducing constrictions of the local vasculature.

Some examples relate to a continuous-flow system for the treatment of edema in an injured central nervous system (CNS) tissue, including:

(a) a reversibly implantable device comprising:

(i) an inflow pathway comprising a first inlet and a first outlet,

(ii) an outflow pathway comprising a second inlet and a second outlet, and

(iii) a fluid flow pathway connecting the first outlet of the inflow pathway and the second inlet of the outflow pathway, wherein the fluid flow pathway comprises a semi-permeable membrane,

(b) a first reservoir;

(c) a fluid-driving apparatus;

(d) a second reservoir; and

(e) a plurality of fluid flow conduits that fluidically connect the first reservoir, the fluid-driving apparatus, the second reservoir, and the reversibly implantable device;

wherein the reversibly implantable device is configured to allow direct contact between the semi-permeable membrane and at least a portion of the injured CNS tissue;

wherein the first reservoir is configured to contain a solution;

wherein the fluid-driving apparatus is configured to pump the solution from the first reservoir, through a conduit, and to the second reservoir;

wherein the second reservoir comprises a vessel and an overflow conduit, such that a head pressure is maintained in the continuous-flow system;

wherein the second reservoir comprises an outlet that is fluidically coupled to the inlet of the inflow pathway of the reversibly implantable device via a fluid flow conduit; and

wherein the solution can pass through the fluid flow pathway, induce osmotic flow of water from the injured CNS tissue across the semipermeable membrane and into the solution, and deliver the water back to the first reservoir.

In some examples, the solution comprises a solute selected from the group of a protein, a carbohydrate, a polysaccharide and a polymer.

In some examples, the semipermeable membrane comprises a material selected from the group consisting of polynephron, polyflux, polysulfone and regenerated cellulose.

In some examples, the semipermeable membrane has a molecular weight cut-off of between about 1 to 60 kilodaltons (kDa).

In some examples, an outer diameter of the fluid flow pathway is 1-2 cm and an inner diameter of the fluid flow pathway is 0.5-1.6 cm.

In some examples, one or more of the inflow pathway comprising a first inlet and a first outlet; the outflow pathway comprising a second inlet and a second outlet and the fluid flow pathway connecting the first outlet of the inflow pathway and the second inlet of the outflow pathway are removably connected to the continuous-flow system.

In some examples, the fluid flow path of the reversibly implantable device, including the semipermeable membrane, conforms to the surface of the traumatically injured CNS tissue.

In some examples, osmotic pressure of the solution is controlled in real time by temperature and/or solute concentration in response to feedback monitoring of a degree of swelling of the CNS tissue, and wherein the system operates on a time scale on the order of a swelling rate to stabilize the tissue.

In some examples, the fluid-driving apparatus is a pump or a gravity feed system.

Some examples relate to a method for removing water from a traumatically injured central nervous system (CNS) tissue in a subject in a controlled fashion, the method including:
(a) exposing a surface of the traumatically injured CNS tissue;
(b) applying a hydrogel to the exposed surface of the traumatically injured CNS tissue, wherein the hydrogel is permeable and allows passage of water,
(c) placing the semipermeable membrane of the reversibly implantable device of the continuous-flow system of claim 1 in contact with the hydrogel; and
(d) flowing or pumping through a lumen of the fluid flow pathway a concentrated solution of a solute that produces a concentration-dependent osmotic pressure, wherein the solute cannot pass through the semi-permeable membrane, wherein the concentrated solution of the solute in the fluid flow pathway of the reversibly implantable device induces an osmotic pressure that draws water from the tissue into the hydrogel and then into the semipermeable membrane, where the water is removed and carried away from the hydrogel and the tissue.

In some examples, the solute is a globular protein.

In some examples, the globular protein is bovine serum albumin (BSA).

In some examples, the BSA is at a concentration of about 350 g/L.

In some examples, the CNS tissue is a spinal tissue.

In some examples, the fluid flow path of the reversibly implantable device, including the semipermeable membrane, conforms to the surface of the traumatically injured CNS tissue.

In some examples, the reversibly implantable device is attached to the subject with an adhesive.

In some examples, a concentration of the solute that cannot pass through the semi-permeable membrane is changed or modified over time to alter the rate of water removal.

In some examples, a concentration of the globular protein that cannot pass through the semi-permeable membrane is altered to between about 0.1 to about 50% to alter the rate of water removal.

In some examples, the pressure of the solution passed across the semi-permeable membrane is altered to change or modify the rate of water removal.

In some examples, the temperature of the concentrated solution is changed in the range of about 20° C. to about 40° C. to alter the rate of water removal.

In some examples, the injured central nervous system (CNS) tissue is associated with the spinal column and the surface of the traumatically injured CNS tissue is exposed by removing or folding back of dorsal processes of vertebra or vertebrae.

In some examples, the hydrogel has a sufficient permeability to allow passage of nutrients, drugs, ions, and water, and wherein the concentrated solution of a solute contains a nutrient, drug or ion.

### BRIEF DESCRIPTION OF THE DRAWINGS

FIG. 1. Simplified illustration of how edema impacts local spinal cord environment and the potential mechanism of action for the OTD. (A) Uninjured tissue has unobstructed vasculature and cerebrospinal fluid (CSF) in the subarachnoid space. (B) After injury, severe edema emanates from the central location of the spinal cord and forces tissue against the dura mater. This constricts local blood vessels in the subarachnoid space (illustrated as red) as well as reduces CSF movement in the local region (Saadoun, S., and Papadopoulos, M. C. (2020). Targeted perfusion therapy in spinal cord trauma. Neurotherapeutics 17, 511-521). (C) The OTD is placed at the point of injury directly on the dura mater. Hydrogel is used to maintain a continuous aqueous interface. The OTD uses osmotic pressure to gently remove water across the permeable tissue (through the cord tissue, pia mater, and dura mater). (D) In time, the OTD may reduce the water content of the swollen tissue to alleviate pressure and constriction of the vasculature and allow fluid movement in the subarachnoid space. Estimates of typical tissue radii with cylindrical approximations for adult rat spinal cord are shown in panel (A) and are derived from the literature (Soubeyrand, M., Badner, A., Vawda, R., Chung, Y. S., and Fehlings, M. G. (2014a). Very high-resolution ultrasound imaging for real-time quantitative visualization of vascular disruption after spinal cord injury. J. Neurotrauma 31, 1767-1775).

FIG. 2. Time course of spinal cord edema following severe SCI at T8 for epicenter. Percent (%) water content calculated in sham rats and injured rats at 1, 6, 12, 24, 48, and 72 h and 5, 7, 14, and 28 days post injury. Error bars indicate standard error (SE). For all times, n=3.

FIG. 3. Time course of edema for rostral (A), and caudal (B).

FIG. 4. Deployment of the osmotic transport device (OTD) to reduce edema at the site of spinal cord injury. (A) Basic OTD design that consists of a fluid housing chamber with a supported semipermeable membrane bottom. The device consists of 19 mm inlet and outlet ports connected by a shallow chamber at the base of the device. The length of the ports allow for ease of access to the device following wound closure. The bottom of the chamber houses the semipermeable membrane (e.g., 10 kDa cutoff) adhered to its underside that provides a continuous flow channel between ports for fluid and solutes not permeating the membrane. The device has an additional silicone housing to protect the animal and provide additional sealing. Images counterclockwise from the top: top view of OTD without silicone housing; bottom view of OTD without membrane attached or silicone housing; bottom view of OTD with attached membrane; bottom view of OTD with membrane attached and silicone housing. (B) Following T8 laminectomy and severe contusion spinal cord injury, the dorsal processes of T7 and T9 are removed and flattened. Hydrogel is then placed on the surface of the exposed injury site and the OTD is deployed and sealed with additional silicone. The hydrogel provides continuous fluid continuity between the OTD semipermeable membrane and the tissue. (C) Photograph of the OTD deployed in the animal without additional silicone application. (D) Aqueous proteinaceous solution (aCSF+BSA, 350 g/L) is delivered from a reservoir (1) through a pump, which is then transferred to a suspended vessel to maintain head pressure (2) via an overflow process (5). The solution is then delivered to the inlet port of the OTD (3) where it passes tangentially across the semipermeable membrane. The BSA is impervious to the membrane and results in an induced osmotic pressure that drives fluid from the tissue into the OTD. The effluent of the OTD is then returned to the beaker (4) where it can once again complete the cycle for continuous treatment.

FIG. 5. Images of the membrane device from the front (A), isometric (B), and from the bottom (C).

FIG. 6. Computational Modeling of Device Efficacy. The membrane device geometry. (A) modelled in COMSOL. The inlet is in the upper left of the geometry and the outlet is in the upper right with the membrane positioned at the middle bottom of the geometry. Meshing, with a maximum mesh size of 1 μm, is shown in (B). Inflow: 25 μL min⁻¹; Protein concentration: 350 g L⁻¹; Membrane hydraulic permeability: 1×10⁻⁷ [m/(s-kPa)]; BSA Diffusion Coefficient: 5.9×10⁻¹¹[m²/s] (Arunyawongsakorn, U., Johnson, C. S., and Gabriel, D. A. (1985). Tracer Diffusion-Coefficients of Proteins by Means of Holographic Relaxation Spectroscopy—Application to Bovine Serum-Albumin. Analytical Biochemistry 146(1), 265-270).

FIG. 7. Evaluation of maximum mesh size to extraction rate at a protein concentration of 350 gL⁻¹ and an inlet flowrate 25 μL min⁻¹. Mesh independence is reached at 1 μm. The maximum mesh size was 1 μm and contained 285,973 degrees of freedom with 5,444 internal degrees of freedom (FIG. 6). Independence from mesh size was determined by evaluating the dependency of extraction rate on maximum mesh size (Table 13). Mesh independence, less than 1% deviation of extraction rate, was determined to occur below 1 μm maximum mesh size.

FIG. 8. Concentration profile of the membrane device given an inflow of 25 μL min⁻¹ and a protein concentration of 350 g L⁻¹. Dilution is noticeable in regions near the membrane of the profiles.

FIG. 9. Computational analysis of fluid extraction rate dependency on inlet flowrate given protein concentration of 350 g L⁻¹. Fluid extraction rate was calculated via the determined velocity through the membrane and the membrane surface area.

FIG. 10. Computational analysis of the average osmotic pressure at the membrane surface dependent on inlet flowrate given protein concentration of 350 g L⁻¹.

FIG. 11. Osmotic pressure data for bovine serum albumin at pH 7.4 in artificial cerebral spinal fluid. An ideal model is shown along with an exponential model fit of the data.

FIG. 12. Effects of OTD treatment on % water content after severe SCI at T8. Percent (%) water content calculated SCI only, SCI+hydrogel (HG), and SCI+OTD rats following 3 h of treatment. The figure shows a statistical reduction in % water content in tissue following OTD treatment. Values are shown for 5 mm segments isolated from the lesion epicenter (n=5 for all groups).

FIG. 13. Calculated illustration of relationship between changes in % water content and potential subarachnoid and vascular compression due to radius swelling. Graphic is based on a typical uninjured cord radius of 1.48 mm (Soubeyrand, M., Badner, A., Vawda, R., Chung, Y. S., and Fehlings, M. G. (2014a). Very high-resolution ultrasound imaging for real-time quantitative visualization of vascular disruption after spinal cord injury. J. Neurotrauma 31, 1767-1775), an overall radius including the subarachnoid space of 1.62 mm (Soubeyrand, M., Badner, A., Vawda, R., Chung, Y. S., and Fehlings, M. G. (2014a). Very high-resolution ultrasound imaging for real-time quantitative visualization of vascular disruption after spinal cord injury. J. Neurotrauma 31, 1767-1775) (radii ratio of 0.913), a 5 mm segment and an uninjured spinal cord water content of 69.4% (normalized as 1.0% water content/initial % water content). Based on measured size of the excised tissue in this study and the assumption of spherical swelling in a cylindrical vessel, a % water content ratio increase of only 0.035 (equivalent to a % water content increase of 71.8%) results in a swelling radius at the threshold for constriction of the subarachnoid space and potential collapse of the local vasculature (radii ratio of 1.0). Although this is only an estimate, nevertheless, these results imply that even relatively small reductions in edema may support reduced vascular compression that may help improve recovery.

FIG. 14. Example impactor analysis showing displacement (microns) vs. time (ms) and Force (K dynes) vs. time (ms).

### DETAILED DESCRIPTION OF THE PREFERRED EMBODIMENT

Spinal cord injuries (SCI) can result in partial or complete loss of sensory function or motor control of the arms, legs or body. In severe cases, SCI can affect bladder and bowel control, breathing, heart rate and blood pressure. Neuropathic pain is a common occurrence following spinal cord injury (SCI), affecting up to 75% of SCI patients (Ahmed et al. 2014 Ann Neurosci 21(3): 97-103). Neuropathic pain is often excruciating and can significantly impact the quality of a patient's life. Dysfunction of the GABAergic system following SCI has been implicated as a mechanism in spinal nocioceptive processing. Reduction of edema in injured spinal tissue following SCI can greatly reduce the pathophysiology of spinal cord injury related to edema.

We disclose a continuous-flow system for the treatment of edema in an injured CNS tissue, including: a reversibly implantable device comprising an inflow pathway, an outflow pathway, and a fluid flow pathway connecting the inlet and the outlet, wherein a semi-permeable membrane rests at least partially in the fluid flow pathway, wherein the device is configured to allow direct contact between the semi-permeable membrane and at least a portion of the injured CNS tissue; a first reservoir; a fluid-driving apparatus; a second reservoir; a plurality of fluid flow conduits that fluidically connect the first reservoir, the fluid-driving apparatus, the second reservoir, and the device, wherein the first reservoir is configured to contain a solution, e.g., a proteinaceous solution comprising BSA, wherein the fluid-driving apparatus is configured to pump the solution from the first reservoir, through a conduit, and to the second reservoir, wherein the second reservoir comprises a suspended vessel and an overflow conduit such that a head pressure is maintained in the system, wherein the second reservoir comprises an outlet that is fluidically coupled to an inflow pathway of the device via a fluid flow conduit, and wherein the solution passes through the semi-permeable membrane and induces osmotic flow of water from the injured CNS tissue into the solution and delivery of the water back to the first reservoir.

### Components of the Continuous-Flow System

The continuous-flow system for the treatment of edema in an injured central nervous system (CNS) tissue, includes: a reversibly implantable device comprising an inflow pathway comprising a first inlet and a first outlet, an outflow pathway comprising a second inlet and a second outlet, and a fluid flow pathway connecting the first outlet of the inflow pathway and the second inlet of the outflow pathway, wherein the fluid flow pathway comprises a semi-permeable membrane. Advantageously, all or portions of the reversibly implantable device, such as the fluid flow pathway in particular, may disposable, and the components of the reversibly implantable device are preferably provided as sterile articles. Other components of the continuous flow system include a first reservoir designed to contain the solution to be circulated through the system, a fluid-driving apparatus; a second reservoir that is optionally connected to an overflow conduit such that a head pressure is maintained in the system; and a plurality of fluid flow conduits that fluidically connect the first reservoir, the fluid-driving apparatus, the second reservoir, and the reversibly implantable device.

### Semipermeable Membrane Selection

The material constituting the semipermeable membrane is not particularly relevant. Rather, the molecular weight cutoff of the semipermeable membrane is what retains solutes in the reversible implantable device and enables water to flow from the hydrated hydrogel and tissue into the lumen of the reversible implantable device.

Transport of fluid through a semi-permeable membrane is governed by physical laws. The flux through a membrane for normal operations follows the Kedem-Katchalsky model. In the Kedem-Katchalsky model, to get flow through a membrane, a pressure applied must be greater than the osmotic pressure. The pressure within the semipermeable membrane is low enough that the flow is reversed and water flows from outside the semipermeable membrane to within the semipermeable membrane.

For example, the following exemplary semipermeable membranes may be used to reduce edema in spinal cord injury models:
Cellulose ester, 5000 MWCO, Molecular/Por, Type C, Spectrum, Laguna Hills, Calif.
Regenerated cellulose, 30 kDa MWCO, NADIR UC030 T, MICRODYN-NADIR, Wiesbaden, Germany.
Regenerated cellulose fibers with a molecular weight cutoff of 13 kDa (132294, Spectrum Laboratories, Inc (Chris Hale Dissertation).
Polysulfone membrane (NADIR® PM UP010, 10 k Da MWCO, Microdyn Nadir, Germany, Wiesbaden).
Polynephron polyethersulfone (PES) membranes (Baxter dialyzer hollow fiber cartridges (XENIUM XPH synthetic fiber devices (110-190).

Each of the above membranes reject the osmotic agent from passage across the membrane, while allowing flow of water across the membrane. An osmotic pressure induced by concentrated solute molecules inside reversibly implantable device causes water outside the semipermeable membrane to be drawn into across the semipermeable membrane and into the reversibly implantable device, thereby reducing edema in spinal cord injury models. In view of the diverse types of semipermeable membranes that we have successfully used, virtually any semipermeable membrane can be used to reduce edema in spinal cord injury models.

### Protein/Polymer Selection

In some examples, proteins and/or dextrans are used to increase the osmolarity of the solution that is circulated through the continuous flow system. Table 1 (below) summarizes exemplary solutes for use in the system. These solutes are selected because of their variation in size, which is coupled to their osmotic pressure. In one embodiment, free-solvent models are used to predict their range of osmotic pressure (using the solvent accessible surface area, and protein charge, as categorized by their isoelectric points, pI). Solutions properties can be selected around the physiological range of cerebrospinal fluid (in mmol/L: Na, 146.5; K, 27.7; Ca²⁺, 1.65; Mg, 1.235; Cl, 213.5, P, 0.65). In one embodiment, viscosity and density of solutions using Ostwald viscometers is determined (e.g., Cannon Fenske Cat. Nos. 75 5560, 150 N956, 200 N843) and a pycnometer (e.g., Kimble Kontes, Cat. No. 15123R-10), respectively.

The solute used in the continuous flow system acts as an osmotic agent in the lumen solution, thereby providing a driving force for water removal from the tissue. Many concentrated solutes and globular proteins (e.g., BSA, or bovine serum albumin) produce non-linear osmotic pressures as concentrations are varied. BSA is merely one example of a solute that can be used to vary osmotic pressure in the presently claimed methods. BSA concentrations over the range of 0-0.3 g/g solution result in osmotic pressures over the range of 0-70 psi. The proteins listed in Table 1 are globular. As such, their physical structures are not expected to change significantly with small changes in solution properties.

TABLE 1
Properties of Selected Proteins

| Protein/Polymer | (kD) | pI | PDB | Ref. |
|---|---|---|---|---|
| Dextrans | ≥60 | | | |
| Hen egg | 14 | 11.0 | 1LZT | [16,, 18] |
| lysozyme (HEL) | | | | |
| Bovine serum | 67 | 4.7 | — | [19] |
| albumin (BSA) | | | | |
| Rabbit | 80 | 7.0 | 1JNF | [20, 22] |
| transferrin | | | | |
| Bovine | 80 | 9.0 | 1LFC | [23, 24] |
| lactoferrin | | | | |

### Hydrogels

Any hydrated hydrogel will work in combination with the continuous flow system disclosed herein. When hydrated, the hydrogel functions as a conduit to conduct water from the injured CNS tissue into the semipermeable membrane. Flow of water into the semipermeable membrane occurs in response to an osmotic pressure across the semipermeable membrane established by the solute in the reversibly implantable device. A person having ordinary skill in the art readily understands that any hydrogel comprising a hydratable, hydrophilic polymeric network that forms three-dimensional crosslinked structures and therefore absorb substantial amounts of water, will work in the continuous flow system disclosed herein. Such hydrogels may include, without limitation, naturally formed hydrogels based on polysaccharides, such as cellulose; natural hydrogels based on polypeptides, such as gelatin; synthetic hydrogels such as a copolymers of N-isopropylacrylamide (NIPAAm) and Jeffamine M-1000 acrylamide (JAAm), poly(methyacrylicgraft-ethylene glycol) (P(MMA-g-EG)), an azobenzen-branched poly(acrylic acid) copolymer, poly(N-isopropylacrylamide) (PNIPAAm), a copolymer of N-isopropylacrylamide (NIPAAm) and itaconic acid (IA), poly(propylene glycol)s (PPG), diepoxy-terminated poly(ethylene glycol)s (PEG), a hydrogel comprising oligo-monomers of poly(ethylene glycol) methyl ether methacrylate, poly(acrylic acid), polymers of N,N-dimethylacrylamide (DMA) or diacetone acrylamide (DAA), Poly (ethylene oxide)-β-poly(propylene oxide)-β-poly (ethylene oxide) triblock copolymers (PEO-PPO-PEO) (known as Pluronic or Poloxamer), poly (hydroxyethyl methacrylate) (pHEMA), Poly Vinyl Alcohol, Polyvinyl alcohol, Starch, Cellulose, Polyethylene, Agarose, chitosan, agar, Guar gum, Gellan gum, Glycol chitosan, Hydroxamated alginates, Alignate bead, Scleroglucan, Poly(acrylic-co-vinylsulfonic) acid, Polyacrylamide and Polyacrylamide/guar gum graft copolymer.

Referring to Table 2 below, we have used agar and DUREPAIR (a commercial product, Dura Regeneration Matrix, Medtronic, Goleta, Calif.) for the hydrated material.

TABLE 2
The Material Properties of the Hydratable Materials. The water contents
were determined by wet-dry weights. The swelling was determined
by comparing the volumes before and after water uptake.

| | | Final Water | | | | |
|---|---|---|---|---|---|---|
| | | Content After | Water Uptake | | | |
| Hydratable | Initial Water | Absorbing | Time | | Rate | |
| Material | Content (%) | Water (%) | (min) | (%) | (%/min) | Swelling (%) |
| 0.3% Agar, | 95.41 ± | N/A | Steady- | 14.93 ± | NA | N/A |
| 3% NaCl | 0.01 | | State | 1.89 | | |
| (n = 2) | | | | | | |
| 0.3% Agar, | 97.86 ± | 98.12 ± 0.15 | 1 | 7.94 ± | 13.24 ± | −10.21 ± |
| aCSF | 0.08 | | | 7.49 | 12.23 | 5.38 |
| (n = 4) | | | 3 | 10.19 ± | 5.67 ± | |
| | | | | 3.85 | 2.11 | |
| | | | 8 | 18.93 ± | 3.94 ± | |
| | | | | 4.05 | 0.80 | |
| 0.4% Agar, | 97.81 ± | 97.80 ± 0.44 | 1 | 5.41 ± | 13.47 ± | 24.40 ± |
| sCSF | 0.12 | | | 1.55 | 4.08 | 4.77 |
| (n = 4) | | | 3 | 11.89 ± | 9.89 ± | |
| | | | | 1.81 | 1.84 | |
| | | | 8 | 21.37 ± | 6.66 ± | |
| | | | | 8.91 | 2.90 | |
| Durepair | 13.30 ± | 84.75 ± 1.46 | 1 | 385.92 ± | 43.38 ± | 17.97 ± |
| (n = 4) | 0.81 | | | 74.65 | 3.40 | 13.77 |
| | | | 3 | 382.81 ± | 14.48 ± | |
| | | | | 41.11 | 1.05 | |
| | | | 8 | 392.44 ± | 5.56 ± | |
| | | | | 70.86 | 0.89 | |

Agar is a jelly-like substance, obtained from red algae. It contains a mixture of two components: the linear polysaccharide agarose, and a heterogeneous mixture of smaller molecules called agaropectin. It forms the supporting structure in the cell walls of certain species of algae. DUREPAIR is a non-synthetic dura substitute for repair of the dura mater during neurosurgical procedures. It uses a strong yet flexible collagen matrix. Both agar and DUREPAIR are hydratable, being able to take up water and to conduct water from one location to another. DUREPAIR performs particularly well with the claimed methods using an osmotic transport device (OTD) since its steady-state water content is only slightly higher than that of injured tissue and it has a large water uptake rate. The optimal hydratable material properties of DUREPAIR provide a rapid initial amelioration of edematous tissue.

In some examples, concentrations of the hydrogel, such as agar (Sigma: A1296-1 kg, CAS: 9002-18-0), in the hydrogel can be with 0.2-3%, for example concentrations of 0.2%, 0.3%, 0.4%, 0.5%, 0.6%, 0.7%, 0.8%, 0.9%, 1.0%, 1.5%, 2.0%, 2.5% and 3.0%. NaCl concentration in the hydrogel is preferably close to a physiological range of the tissue, such as 2-5%, for example concentrations of 2.0%, 2.5%, 3.0%, 3.5%, 4.0%, 4.5% and 5.0%. Hydraulic permeability is determined prior to use.

### Osmotic Pressure and Solutes

When a semipermeable membrane separates a solvent, such as water, and a solution containing an impermeable solute, a net flow of the solvent occurs from the solvent to the solution attempting to dilute the solution. The net flow is a phenomenon called osmosis.

Various solutes (e.g., polymers, such as carbohydrates and proteins) may be dissolved in water to produce an aqueous solution within the continuous flow system, which results in an osmotic pressure in aqueous media outside the semipermeable membrane that draws water from the tissue/hydrogel into the reversibly implantable device.

An entire ultrafiltration industry is based on membrane separations technology and osmotic pressure generated by various types of osmolytes. Osmotic pressure can be generated by a variety of solutes, including various proteins (e.g., Hen egg lysozyme (HEL), Bovine Immuno-gamma Globulin (IgG), α-Crystallin; and other large molecules such as dextrose and sucrose, as non-limiting examples.

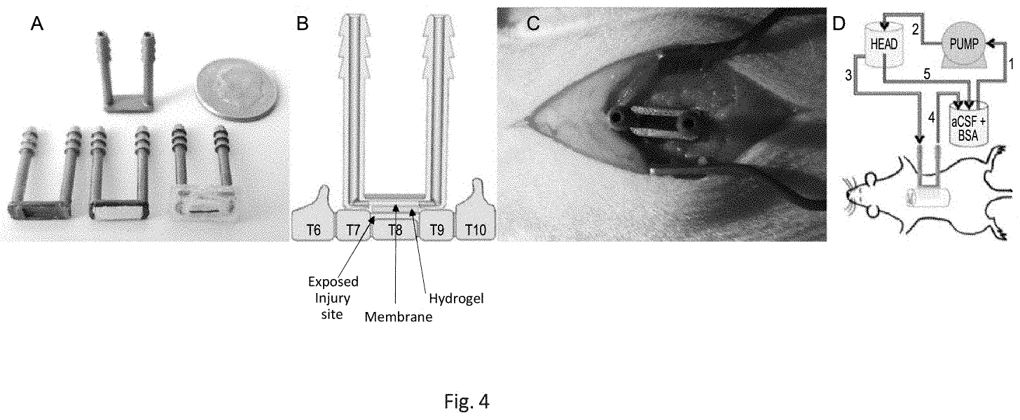

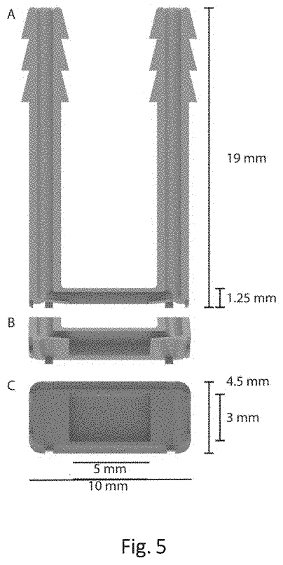

In some examples, the solution contained within the continuous flow system comprises a concentrated protein, carbohydrate, polysaccharide or polymer solution, or osmolyte solution or rejected solute, wherein the solution containing the concentrated protein, carbohydrate, polysaccharide or polymer solution, or concentrated osmolyte solution or rejected solute passes through the lumen of the semi-permeable membrane, and the concentrated protein carbohydrate, polysaccharide or polymer or concentrated osmolyte solution or rejected solute induces an osmotic pressure that drives water into the reversibly implantable device where it is removed and carried away from the edematous tissue. An exemplary device of the invention is illustrated in FIGS. 4 and 5, as discussed in detail, below.

In some embodiments, the solution or lumen contents further comprise nutrients, or drugs, and optionally the drugs and/or nutrients are for the treatment or amelioration of the edema, or injury, or an underlying disease or condition causing the edema, and optionally the drugs comprise or are small molecules or proteins, and optionally the drugs act as antibiotics, anti-inflammatories, vasoconstrictors, vascular or tissue growth stimulating agents;

In some examples, an aqueous proteinaceous, carbohydrate or polysaccharide solution is flowed (e.g., by osmotic force) or is flowed or pumped or passively flows (such as head pressure) through the reversibly implantable device.

In some examples, the semipermeable membrane completely or substantially rejects a solute but allows (relatively) easy passage of ions, electrolytes and water, and also nutrients (such as oxygen or glucose) and small molecules, proteins and other drugs,

In some examples, a hydrogel or an equivalent gel (e.g., a hydrophilic gel) is used to maintain a membrane-tissue contact.

In some examples, the temperature of the lumen solution is about 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C. or within a range having upper and lower limits defined by any of the preceding values.

In some examples, the hydrogel has a sufficient permeability to allow (relatively) easy passage of nutrients, drugs, ions, and water.

In some examples, the hydrogel used is rigid enough to maintain membrane-tissue contact and to support the reversibly implantable device.

In some examples an outer diameter of the fluid flow pathway that comprises the semipermeable membrane is between about 1 mm and 2 cm, including diameters of 1 mm, 2 mm, 3 mm, 4 mm, 5 mm, 6 mm, 7 mm, 8 mm, 9 mm, 1 cm, 1.1 cm, 1.2 cm, 1.3 cm, 1.4 cm and 1.5 cm, 1.6 cm, 1.7 cm, 1.8 cm and 2.0 cm. In some examples, the inner diameter of the fluid flow pathway has an inner diameter of between about 0.5 mm and 1.6 cm, including diameters of 1 mm, 2 mm, 3 mm, 4 mm, 5 mm, 6 mm, 7 mm, 8 mm, 9 mm, 1 cm, 1.1 cm, 1.2 cm, 1.3 cm, 1.5 cm and 1.6 cm.

In some examples, the semipermeable membrane comprises cellulose fibers, regenerated cellulose, a biocompatible material, or a bioinert material.

In some examples, the semipermeable membrane has a molecular weight cut-off of less than about 100 daltons for a rejected carbohydrate or a rejected salt. In other examples, the semipermeable membrane has a molecular weight cut-off of between about 100 to about 1000 Daltons for a carbohydrate or a polymer solution. In other examples, the semipermeable membrane has a molecular weight cut-off of between about 1 to about 60 kDa or greater than about 60 kDa. In other examples, the molecular weight cut-off is 5 to 20 kilodalton (kDa), between about 1 to 30 kDa, or about 5, 6, 7, 8, 9, 10, 11, 12, 13, 14, 15, 16, 17, 18, 19 or 20 or more kDa.

In some examples, the semipermeable membrane permits reverse osmosis.

In some examples, the continuous-flow system provides methods for: removing a fluid or a water from an edematous tissue, e.g., a central nervous system (CNS) tissue, or a spinal or a brain tissue, or an injured, insulted (e.g., by chemical exposure) or burned tissue, in a controlled fashion, or removing a fluid or a water from an edematous area, e.g., a central nervous system (CNS) tissue, a spinal or a brain tissue, or an injured, insulted (e.g., by chemical exposure) or burned tissue, in a controlled fashion to treat the edema, e.g., the central nervous system (CNS) edema, or a spinal or a cerebral edema; or treat or reverse a CNS, spinal or a brain inflammation or a CNS, spinal or a brain injury, or an inflammation due to an injury, a chemical exposure or a trauma.

In some examples, the reversibly implantable device is attached to a subject using an adhesive, such as silicon, surgical glue or adhesive tape.

Some examples provide portable or small kits comprising a continuous flow system as disclosed herein and an associated gel, wherein the gel is optionally a hydrogel.

In some examples, osmotic pressure is controlled by temperature, concentration, and/or solute. Because the osmotic pressure is generated by the presence of the rejected species in the device lumen and not the ion species, the process has the advantage of maintaining ionic equilibrium. Furthermore, the system will operate in time scales on the order of the swelling rate, ensuring system stability and allowing effective feedback control. In some examples, scattering information from optical coherence tomography (OCT) is used to infer swelling rate in the feedback process. The invention provides an integrated system to detect and reverse edema and, thus reduce complications due to a CNS injury, such as an SCI in affected individuals.

The continuous flow system exploits the inevitable osmotic pressure that is generated during transport of concentrated rejected species (such as proteins or polymers) across a semi-permeable membrane in the presence of aqueous systems. Typically, membrane processes are used to separate or exchange solutes in the media in contact with the membrane. In doing so, the permeate flux is limited due to the osmotic pressure of the rejected solutes due to concentration polarization. As an example, one of the most common models used to relate permeate flux, J, to the transmembrane pressure driving force, ΔP, is the Kedem-Katchalsky model which states that:

$$J = \frac{Δ P - σΔπ}{μ ( {R}_{m} + {R}_{p} )}$$

The osmotic pressure, Δπ, is a function of the solute concentration difference across the pores at the membrane surface. The osmotic reflection coefficient, σ, provides a measure of the membrane permselectivity, Rm is the membrane resistance during ultrafiltration, Rp is the extra resistance associated with any fouling, and μ is the solution viscosity. The osmotic pressure in these processes is largely regarded as a resistance to separation and must be overcome by increasing the operating transmembrane pressure. For a hollow fiber or bundle/module device, the transmembrane pressure is an average of the hydraulic pressure in the lumen minus the pressure on the outside of the fiber.

In some examples, the methods involve: (a) applying a permeable, non-rigid hydrogel, soft hydrogel or gel to an exposed surface of a tissue, wherein the permeable, non-rigid hydrogel, soft hydrogel or gel substantially conforms to the tissue to maximize contact area with the tissue, and (b) placing the reversibly implantable device including a semipermeable membrane in contact with the permeable, non-rigid hydrogel, soft hydrogel or gel, and (c) wherein the concentrated solution of the protein, carbohydrate, polymer or the solute in the hollow fiber induces an osmotic pressure that draws water from the tissue into the permeable, non-rigid hydrogel, soft hydrogel or gel and then into the hollow fiber membrane, where the water is removed and carried away from the permeable, non-rigid hydrogel, soft hydrogel or gel and the tissue.

In one embodiment, the device of the invention can actively remove water from a CNS, spinal or brain tissue in vivo, which can be demonstrated in animal models of spinal edema, for example.

In some examples, the continuous-flow system is portable, and advantages of the portable design, or “portability”, is its use in the event of a catastrophic event or in the warfare theatre during active combat.

In some, the design for flow-through the lumen (the protein solution) can be achieved with very low flow including gravity feed. In some examples, a cerebrospinal fluid (CSF) solution can be stored in flexible bags (just as lactated ringer's solution or saline solutions used in hospitals and temporary combat emergency facilities such as MASH (Mobile Army Surgical Hospital). In some examples, these bags are connected to transfer tubing and the device and hung over the patient's injury, the resulting flow will be sufficient to induce the osmotic pressure effect. In the field, the device design will be effective for first responders, and can be carried in a small kit that supplies the tubing, hollow fiber device and the associated gel. The kit can be rapidly deployed and the flexibility of the fibers, as well as the efficacy of the device (it does not need to cover the entire edematous, injured area), allows for its use in a number of emergency applications such as spinal swelling.

### Kits and Instructions

Also disclosed are kits for use in combination with a continuous flow system disclosed herein, for example comprising hydrogel, solute(s) such as protein and polymer, and components of the continuous flow system such as an inflow pathway comprising a first inlet and a first outlet, an outflow pathway comprising a second inlet and a second outlet, a reusable or disposable fluid flow pathway configured to connect to the first outlet of the inflow pathway and the second inlet of the outflow pathway, wherein the fluid flow pathway comprises a semi-permeable membrane, a first reservoir; a fluid-driving apparatus; a second reservoir; and a plurality of fluid flow conduits that fluidically connect the first reservoir, the fluid-driving apparatus, the second reservoir, and the reversibly implantable device. In some examples, the kit contains and instructions for use of the continuous flow system and various elements thereof.

### EXAMPLE

### Implantable Osmotic Transport Device can Reduce Edema after Severe Contusion Spinal Cord Injury

Recent findings from the injured spinal cord pressure evaluation (ISCoPE) study indicate that, after severe contusion to the spinal cord, edema originating in the spinal cord accumulates and compresses the tissue against the surrounding dura mater, despite decompressive laminectomy. It is hypothesized that this compression results in restricted flow of cerebrospinal fluid (CSF) in the subarachnoid space and central canal and ultimately collapses local vasculature, exacerbating ischemia and secondary injury. Here we developed a surgically mounted osmotic transport device (OTD) that rests on the dura and can osmotically remove excess fluid at the injury site. Tests were performed in 4-h studies immediately following severe (250 kD) contusion at T8 in rats using the OTD. A 3-h treatment with the OTD after 1-h post injury significantly reduced spinal cord edema compared to injured controls. A first approximation mathematical interpretation indicates that this modest reduction in edema provides a basis to relieve compression of local vasculature and restore flow of CSF in the region. In addition, we determined the progression of edema up to 28 days after insult in the rat for the same injury model. Results showed peak edema at 72 h. These results indicate that incorporating the OTD to operate continuously at the site of injury throughout the critical period of edema progression provides a basis for significant improvement of recovery following contusion spinal cord injury.

### Results

Progression of Edema after Severe Contusion at T8

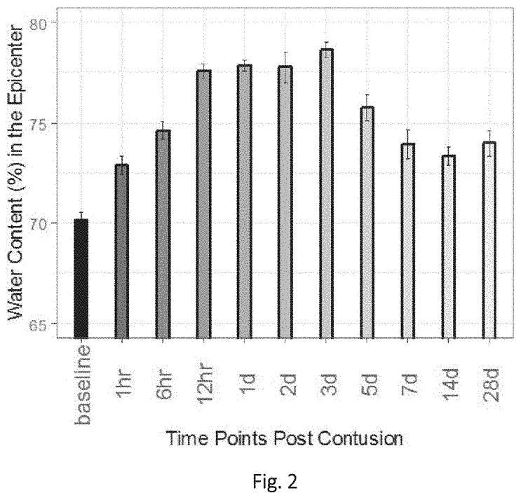

We examined edema progression (percent water content) at 1, 6, 12, 24, 48, 72 h and 5, 7, 14, and 28 days (d) after injury. FIG. 2 shows the resulting % water content results at the epicenter. It is seen that water content in the epicenter increases immediately in an hour after contusion. It approaches its peak on day 3 or 72 h post contusion. By the end of the study on day 28, water content is still very higher compared to that at baseline. The numerical values are shown in Table 3. These differences are tested using a linear mixed model (Cnaan, A., Laird, N. M., and Slasor, P. (1997). Using the general linear mixed model to analyze unbalanced repeated measures and longitudinal data. Stat. Med. 16, 2349-2380; Festing, M. F., and Altman, D. G. (2002). Guidelines for the design and statistical analysis of experiments using laboratory animals. ILAR J. 43, 244-258). It is shown that water content is significantly higher compared to baseline values at 1 h, 3 days and 28 days after contusion (2.73, 8.50, and 3.80%, respectively) (Table 4).

TABLE 3
Time Course for Edema at Epicenter

| | Sample | | | |
|---|---|---|---|---|
| Time points | size | Mean | SD | SE |
| Baseline | 3 | 70.17 | 0.61 | 0.35 |

| | | | | | |
|---|---|---|---|---|---|
| 1 | hr | 3 | 72.90 | 0.79 | 0.46 |
| 6 | hr | 3 | 74.60 | 0.72 | 0.42 |
| 12 | hr | 3 | 77.57 | 0.64 | 0.37 |
| 1 | d | 3 | 77.87 | 0.42 | 0.24 |
| 2 | d | 3 | 77.77 | 1.33 | 0.77 |
| 3 | d | 3 | 78.67 | 0.67 | 0.38 |
| 5 | d | 3 | 75.77 | 1.18 | 0.68 |
| 7 | d | 3 | 73.93 | 1.22 | 0.71 |
| 14 | d | 3 | 73.37 | 0.81 | 0.47 |
| 28 | d | 3 | 73.97 | 1.10 | 0.64 |

TABLE 4
Water content comparisons at the four critical stages

| Critical time point | Water content or | | |
|---|---|---|---|
| comparison | its difference (%) | 95% CI | P value |
| Baseline | 70.17 | 69.01-71.32 | <0.0001 |
| Post 1 h vs | 2.73 | 1.34-4.13 | 0.003 |
| Baseline | | | |
| Post 3 days vs | 8.50 | 7.11-9.89 | <0.0001 |
| Baseline | | | |
| Post 28 days vs | 3.80 | 2.41-5.19 | 0.0005 |
| Baseline | | | |

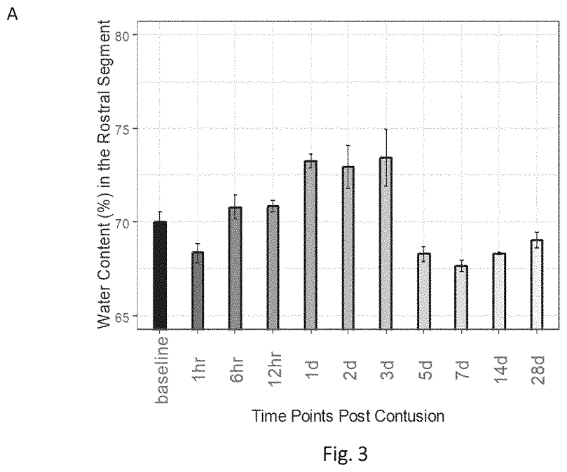

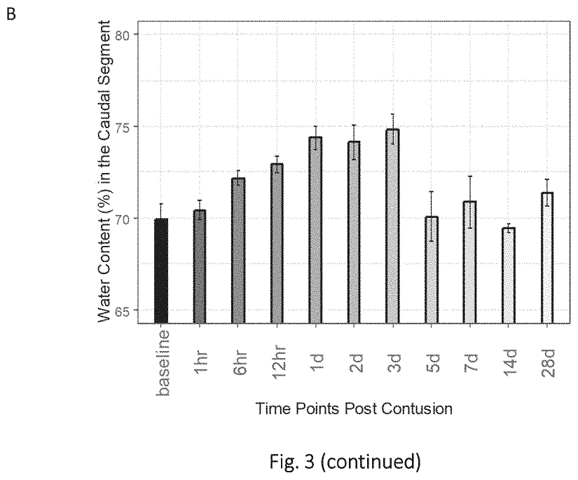

In addition, rostral and caudal areas adjacent to the lesion epicenter showed significant increases in water content 24 h after injury, peaking at 72 h before returning to baseline at 7 d. However, water content was only different from its baseline on day 3 after contusion in the rostral and caudal segments. (Rostral and caudal time course FIG. 3 and Tables 3-6.) Raw data is available in Table 9.

TABLE 5
Water content summary in the rostral segment

| | Sample | | | |
|---|---|---|---|---|
| Time points | size | Mean | sd | se |
| Baseline | 3 | 69.97 | 0.97 | 0.56 |

| | | | | | |
|---|---|---|---|---|---|
| 1 | hr | 3 | 68.33 | 0.86 | 0.50 |
| 6 | hr | 3 | 70.80 | 1.11 | 0.64 |
| 12 | hr | 3 | 70.83 | 0.57 | 0.33 |
| 1 | d | 3 | 73.23 | 0.64 | 0.37 |
| 2 | d | 3 | 72.97 | 2.00 | 1.16 |
| 3 | d | 3 | 73.43 | 2.65 | 1.53 |
| 5 | d | 3 | 68.27 | 0.72 | 0.42 |
| 7 | d | 3 | 67.63 | 0.57 | 0.33 |
| 14 | d | 3 | 68.30 | 0.10 | 0.06 |
| 28 | d | 3 | 69.00 | 0.72 | 0.42 |

TABLE 6
Water content summary in the caudal segment

| Time | Sample | | | |
|---|---|---|---|---|
| points | size | Mean | sd | se |
| Baseline | 3 | 69.90 | 1.51 | 0.87 |

| | | | | | |
|---|---|---|---|---|---|
| 1 | hr | 3 | 70.43 | 0.85 | 0.49 |
| 6 | hr | 3 | 72.17 | 0.68 | 0.39 |
| 12 | hr | 3 | 72.93 | 0.78 | 0.45 |
| 1 | d | 3 | 74.37 | 1.12 | 0.64 |
| 2 | d | 3 | 74.13 | 1.59 | 0.92 |
| 3 | d | 3 | 74.83 | 1.44 | 0.83 |
| 5 | d | 3 | 70.07 | 2.34 | 1.35 |
| 7 | d | 3 | 70.87 | 2.49 | 1.44 |
| 14 | d | 3 | 69.43 | 0.45 | 0.26 |
| 28 | d | 3 | 71.37 | 1.27 | 0.73 |

TABLE 7
Water content comparisons in the rostral
segment at the 4 critical stages

| Critical time point | Water content or | | |
|---|---|---|---|
| comparison | its difference (%) | 95% CI | P value |
| Baseline | 69.97 | 69.01-71.32 | <0.0001 |
| Post 1 hr. vs. | −1.63 | −4.66-1.40 | 0.24 |
| Baseline | | | |
| Post 3 days vs. | 3.47 | 0.44-6.50 | 0.03 |
| Baseline | | | |
| Post 28 days vs. | −0.97 | −4.00-2.06 | 0.46 |
| Baseline | | | |

TABLE 8
Water content comparisons in the caudal
segment at the 4 critical stages

| Critical time point | Water content or | | |
|---|---|---|---|
| comparison | its difference (%) | 95% CI | P value |
| Baseline | 69.90 | 68.08-71.72 | <0.0001 |
| Post 1 hr. vs. | 0.53 | −2.05-3.11 | 0.63 |
| Baseline | | | |
| Post 3 days vs. | 4.93 | 2.35-7.51 | 0.003 |
| Baseline | | | |
| Post 28 days vs. | 1.47 | −1.11-4.05 | 0.21 |
| Baseline | | | |

TABLE 9
Data used in edema progression analysis

| | | 3 | 13 | 14 | 20 | 21 | 24 | 19 | 22 | 23 | 25 | 27 |
|---|---|---|---|---|---|---|---|---|---|---|---|---|
| ANIMAL GROUP | | sham | sham | sham | 250kD | 250kD | 250kD | 250kD | 250kD | 250kD | 250kD | 250kD |
| TIME POINT | | 24 hr | 24 hr | 24 hr | 1 hr | 1 hr | 1 hr | 6 hr | 6 hr | 6 hr | 12 hr | 12 hr |
| Epicenter | Foil | 97.1 | 52.7 | 63.2 | 56.1 | 57.9 | 45 | 43 | 52.1 | 57.1 | 68.2 | 61.6 |
| | Wet + | 116.6 | 70.4 | 85.4 | 76.7 | 80.9 | 71.3 | 65.5 | 77.5 | 77.3 | 95.1 | 93.8 |
| | Foil | | | | | | | | | | | |
| | Dry + | 102.9 | 58.1 | 69.7 | 61.8 | 64.2 | 51.9 | 48.9 | 58.4 | 62.2 | 74.3 | 68.6 |
| | Foil | | | | | | | | | | | |
| | Wet | 19.5 | 17.7 | 22.2 | 20.6 | 23 | 26.3 | 22.5 | 25.4 | 20.2 | 26.9 | 32.2 |
| | Dry | 5.8 | 5.4 | 6.5 | 5.7 | 6.3 | 6.9 | 5.9 | 6.3 | 5.1 | 6.1 | 7 |
| | Percent | 70.3% | 69.5% | 70.7% | 72.3% | 72.6% | 73.8% | 73.8% | 75.2% | 74.8% | 77.3% | 78.3% |
| Rostral | Foil | 85.1 | 53.8 | 60.8 | 51.5 | 57.5 | 42.8 | 37 | 61.9 | 57.2 | 77.3 | 66.6 |
| | Wet | 105.6 | 74.7 | 81.7 | 74.8 | 80.5 | 66.6 | 63 | 86 | 84.5 | 101.8 | 93.5 |
| | Dry | 91.2 | 60.3 | 66.9 | 59.1 | 64.6 | 50.3 | 44.9 | 68.9 | 64.9 | 84.6 | 74.4 |
| | Wet | 20.5 | 20.9 | 20.9 | 23.3 | 23 | 23.8 | 26 | 24.1 | 27.3 | 24.5 | 26.9 |
| | Dry | 6.1 | 6.5 | 6.1 | 7.6 | 7.1 | 7.5 | 7.9 | 7 | 7.7 | 7.3 | 7.8 |
| | Percent | 70.2% | 68.9% | 70.8% | 67.4% | 69.1% | 68.5% | 69.6% | 71.0% | 71.8% | 70.2% | 71.0% |
| Caudal | Foil | 81.5 | 57.4 | 67.6 | 56 | 60.7 | 54.1 | 50.9 | 53.3 | 52 | 76.4 | 56.8 |
| | Wet | 105.3 | 80.9 | 91.7 | 81.2 | 81.1 | 86.5 | 78.4 | 82.7 | 82.4 | 108.4 | 92 |
| | Dry | 89 | 64.1 | 74.9 | 63.2 | 66.8 | 63.9 | 58.4 | 61.7 | 60.4 | 84.8 | 66.4 |
| | Wet | 23.8 | 23.5 | 24.1 | 25.2 | 20.4 | 32.4 | 27.5 | 29.4 | 30.4 | 32 | 35.2 |
| | Dry | 7.5 | 6.7 | 7.3 | 7.2 | 6.1 | 9.8 | 7.5 | 8.4 | 8.4 | 8.4 | 9.6 |
| | Percent | 68.5% | 71.5% | 69.7% | 71.4% | 70.1% | 69.8% | 72.7% | 71.4% | 72.4% | 73.8% | 72.7% |

| | | 28 | 4 | 7 | 8 | 16 | 18 | 44 | 9 | 11 | 12 |
|---|---|---|---|---|---|---|---|---|---|---|---|
| ANIMAL GROUP | | 250kD | 250kD | 250kD | 250kD | 250kD | 250kD | 250kD | 250kD | 250kD | 250kD |
| TIME POINT | | 12 hr | 24 hr | 24 hr | 24 hr | 48 hr | 48 hr | 248 hr | 72 hr | 72 hr | 72 hr |
| Epicenter | Foil | 64.4 | 82.2 | 63.1 | 74.5 | 52.5 | 53.8 | 66.8 | 55.3 | 49.5 | 57 |
| | Wet + | 95.4 | 107 | 87.6 | 95.7 | 77.1 | 77.7 | 95.1 | 78.8 | 74.3 | 83.3 |
| | Foil | | | | | | | | | | |
| | Dry + | 71.5 | 87.6 | 68.5 | 79.3 | 57.6 | 59.3 | 73.3 | 60.5 | 54.7 | 62.5 |
| | Foil | | | | | | | | | | |
| | Wet | 31 | 24.8 | 24.5 | 21.2 | 24.6 | 23.9 | 28.3 | 23.5 | 24.8 | 26.3 |
| | Dry | 7.1 | 5.4 | 5.4 | 4.8 | 5.1 | 5.5 | 6.5 | 5.2 | 5.2 | 5.5 |
| | Percent | 77.1% | 78.2% | 78.0% | 77.4% | 79.3% | 77.0% | 77.0% | 77.9% | 79.0% | 79.1% |
| Rostral | Foil | 53.5 | 86.1 | 61.8 | 58.8 | 46.7 | 55.1 | 64.4 | 50.9 | 46.5 | 51.7 |
| | Wet | 83.5 | 109.3 | 85.4 | 81.8 | 68.6 | 80.4 | 89.1 | 72.8 | 65.3 | 77.3 |
| | Dry | 62.1 | 92.2 | 68.3 | 64.9 | 52.2 | 61.9 | 71.6 | 57.3 | 51 | 58.5 |
| | Wet | 30 | 23.2 | 23.6 | 23 | 21.9 | 25.3 | 24.7 | 21.9 | 18.8 | 25.6 |
| | Dry | 8.6 | 6.1 | 6.5 | 6.1 | 5.5 | 6.8 | 7.2 | 6.4 | 4.5 | 6.8 |
| | Percent | 71.3% | 73.7% | 72.5% | 73.5% | 74.9% | 73.1% | 70.9% | 70.8% | 76.1% | 73.4% |
| Caudal | Foil | 60.5 | 66.3 | 69.8 | 64.5 | 57 | 59 | 73.6 | 55.8 | 59.5 | 55 |
| | Wet | 95.5 | 94.1 | 100.6 | 92.3 | 86 | 86.1 | 107.5 | 81.5 | 88.5 | 83.9 |
| | Dry | 70.2 | 73.3 | 78.1 | 71.4 | 64.2 | 65.8 | 83 | 62.7 | 66.5 | 62.1 |
| | Wet | 35 | 27.8 | 30.8 | 27.8 | 29 | 27.1 | 33.9 | 25.7 | 29 | 28.9 |
| | Dry | 9.7 | 7 | 8.3 | 6.9 | 7.2 | 6.8 | 9.4 | 6.9 | 7 | 7.1 |
| | Percent | 72.3% | 74.8% | 73.1% | 75.2% | 75.2% | 74.9% | 72.3% | 73.2% | 75.9% | 75.4% |

| | | 47 | 48 | 49 | 30 | 31 | 50 | 34 | 35 | 40 |
|---|---|---|---|---|---|---|---|---|---|---|
| ANIMAL GROUP | | 250kD | 250kD | 250kD | 250kD | 250kD | 250kD | 250kD | 250kD | 250kD |
| TIME POINT | | 5 d | 5 d | 5 d | 7 d | 7 d | 7 d | 14 d | 14 d | 14 d |
| Epicenter | Foil | 55.5 | 50.8 | 57.8 | 55.9 | 67.9 | 60.8 | 64.7 | 63.1 | 67.8 |
| | Wet + | 81 | 73.1 | 84.1 | 80.7 | 88.8 | 84.8 | 89.4 | 84.2 | 86 |
| | Foil | | | | | | | | | |
| | Dry + | 61.5 | 56.5 | 64 | 62.7 | 73.3 | 66.8 | 71.1 | 68.7 | 72.8 |
| | Foil | | | | | | | | | |
| | Wet | 25.5 | 22.3 | 26.3 | 24.8 | 20.9 | 24 | 24.7 | 21.1 | 18.2 |
| | Dry | 6 | 5.7 | 6.2 | 6.8 | 5.4 | 6 | 6.4 | 5.6 | 5 |
| | Percent | 76.5% | 74.4% | 76.4% | 72.6% | 74.2% | 75.0% | 74.1% | 73.5% | 72.5% |
| Rostral | Foil | 54.5 | 54.3 | 62 | 59.4 | 61.8 | 63.4 | 68.9 | 64.1 | 67.5 |
| | Wet | 78.1 | 78 | 87.8 | 82.9 | 85.7 | 85.2 | 93.3 | 90.3 | 86.7 |
| | Dry | 61.8 | 61.9 | 70.3 | 66.9 | 69.5 | 70.6 | 76.6 | 72.4 | 73.6 |
| | Wet | 23.6 | 23.7 | 25.8 | 23.5 | 23.9 | 21.8 | 24.4 | 26.2 | 19.2 |
| | Dry | 7.3 | 7.6 | 8.3 | 7.5 | 7.7 | 7.2 | 7.7 | 8.3 | 6.1 |
| | Percent | 69.1% | 67.9% | 67.8% | 68.1% | 67.8% | 67.0% | 68.4% | 68.3% | 68.2% |
| Caudal | Foil | 55.3 | 49.9 | 59 | 57.8 | 64.7 | 57 | 69.4 | 65.4 | 61.1 |
| | Wet | 83.9 | 83.1 | 89.9 | 82.9 | 93.4 | 87.3 | 100.8 | 94.3 | 88.8 |
| | Dry | 64 | 59 | 68.9 | 64.8 | 72.6 | 66.7 | 79 | 74.1 | 69.7 |
| | Wet | 28.6 | 33.2 | 30.9 | 25.1 | 28.7 | 30.3 | 31.4 | 28.9 | 27.7 |
| | Dry | 8.7 | 9.1 | 9.9 | 7 | 7.9 | 9.7 | 9.6 | 8.7 | 8.6 |
| | Percent | 69.6% | 72.6% | 68.0% | 72.1% | 72.5% | 68.0% | 69.4% | 69.9% | 69.0% |

| | | 39 | 41 | 45 | 15 | 17 | 26 | 29 | 42 |
|---|---|---|---|---|---|---|---|---|---|
| ANIMAL GROUP | | 250kD | 250kD | 250kD | 250kD | 250kD | 250kD | 250kD | 250kD |
| TIME POINT | | 28 d | 28 d | 28 d | 48 hr | 48 hr | 12 hr | 7 d | 28 d |
| Epicenter | Foil | 64.5 | 65.4 | 56.8 | 58 | 51.3 | 68.7 | 57.4 | 63.8 |
| | Wet + | 82.7 | 82.6 | 72.5 | 81.4 | 75.7 | 90.4 | 84 | 74.9 |
| | Foil | | | | | | | | |
| | Dry + | 69.1 | 70.1 | 60.8 | 63.8 | 56.6 | 74.4 | 65 | 66 |
| | Foil | | | | | | | | |
| | Wet | 18.2 | 17.2 | 15.7 | 23.4 | 24.4 | 21.7 | 26.6 | 11.1 |
| | Dry | 4.6 | 4.7 | 4 | 5.8 | 5.3 | 5.7 | 7.6 | 2.2 |
| | Percent | 74.7% | 72.7% | 74.5% | 75.21% | 78.28% | 73.73% | 71.43% | 80.18% |
| Rostral | Foil | 63.3 | 67.4 | 52.8 | 44.5 | 52 | 67 | 55.4 | 65.7 |
| | Wet | 85.4 | 92.4 | 73.9 | 66.9 | 74.7 | 87 | 77.6 | 87.9 |
| | Dry | 70.1 | 75 | 59.5 | 51.4 | 58 | 73.1 | 61.1 | 72.7 |
| | Wet | 22.1 | 25 | 21.1 | 22.4 | 22.7 | 20 | 22.2 | 22.2 |
| | Dry | 6.8 | 7.6 | 6.7 | 6.9 | 6 | 6.1 | 5.7 | 7 |
| | Percent | 69.2% | 69.6% | 68.2% | 69.20% | 73.57% | 69.50% | 74.32% | 68.47% |
| Caudal | Foil | 68.4 | 65.4 | 51.2 | 55.9 | 62.7 | 70.6 | 62.9 | 67.9 |
| | Wet | 94.9 | 91.9 | 72.5 | 84.6 | 93.1 | 99.5 | 90 | 91.8 |
| | Dry | 75.6 | 73.1 | 57.5 | 64.3 | 70 | 78.7 | 70.8 | 74.7 |
| | Wet | 26.5 | 26.5 | 21.3 | 28.7 | 30.4 | 28.9 | 27.1 | 23.9 |
| | Dry | 7.2 | 7.7 | 6.3 | 8.4 | 7.3 | 8.1 | 7.9 | 6.8 |
| | Percent | 72.8% | 70.9% | 70.4% | 70.73% | 75.99% | 71.97% | 70.85% | 71.55% |

### Development of a Spinal Cord Osmotic Transport Device

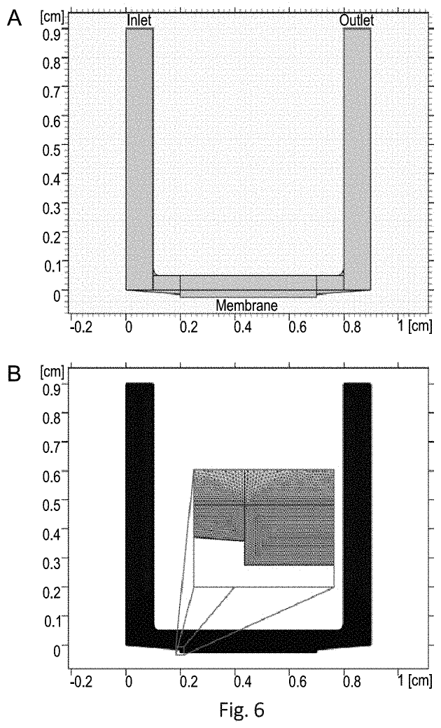

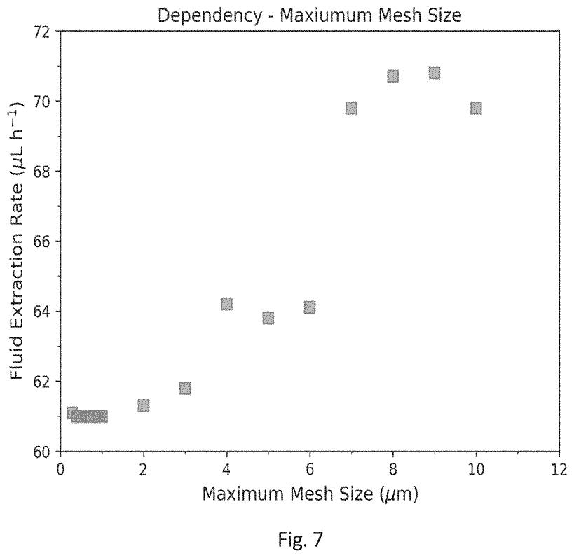

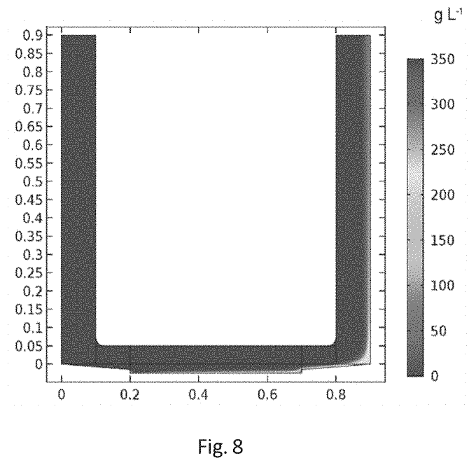

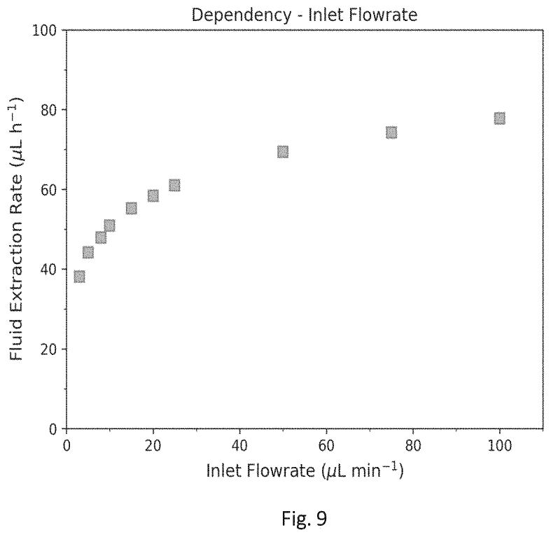

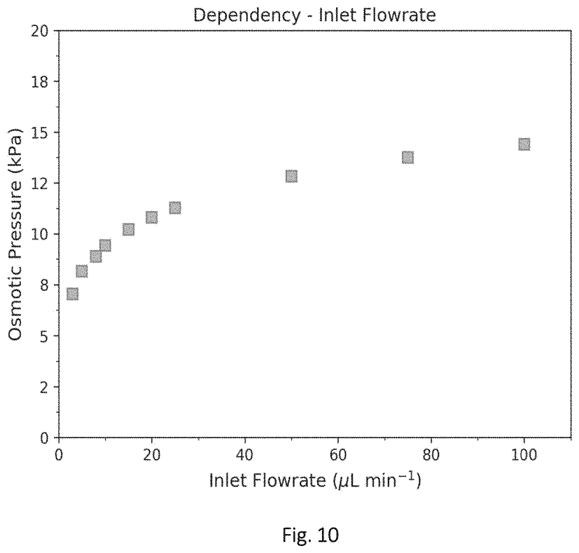

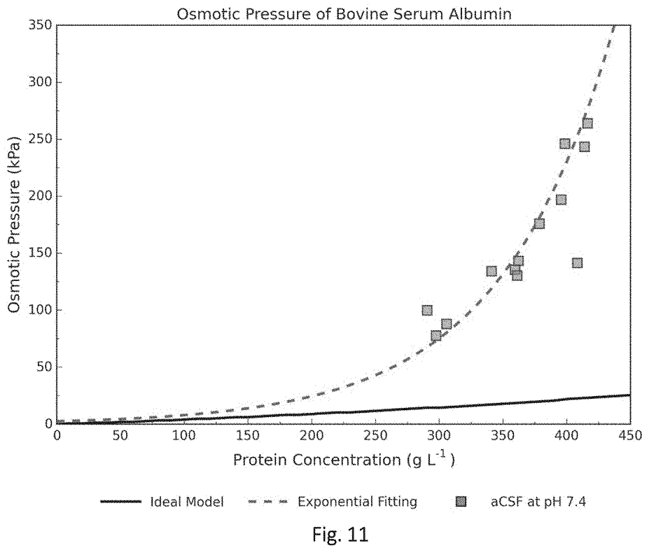

The device design consists of a flat semi-permeable membrane separations structure that is mounted in a two-compartment housing with two ports that allow tangential flow of an osmotically active fluid across the membrane on one side (FIG. 4, A). The osmolyte is impervious to the membrane but water and ions can freely cross the barrier. The opposite side of the membrane is loaded with a hydrogel and is placed direct in contact with the tissue at the point of injury (FIG. 4, B) in the animal (FIG. 4, C). Images of the membrane device design are shown in FIG. 5. Computational modeling of the membrane device efficacy is shown in FIG. 6. Concentration profile of the membrane device is shown in FIG. 8, which demonstrates that dilution is significant near the membrane. We demonstrate that fluid extraction rate (FIG. 9) and average osmotic pressure (FIG. 10) are dependent on inlet flowrate. Artificial cerebral spinal fluid (aCSF) containing 350 g/L bovine serum albumin (BSA) as the osmolyte is circulated through the device (FIG. 4, D) for 3 h beginning 1 h after injury. The device is estimated to have an extraction rate on the order of 30 mL/h (see Table 10). Following treatment, the animal is sacrificed, and tissue is dissected for analysis of spinal cord % water content.

TABLE 10
Simulated Extraction Rate Dependence on Inlet Flowrate
for Membrane Device with Bovine Serum Albumin in Artificial
Cerebral Spinal Fluid at pH 7.4, 25° C.

| Inlet Flowrate | Extraction Rate |
|---|---|
| (μL min⁻¹) | (μL h⁻¹) |
| 3 | 38.1 |
| 5 | 44.1 |
| 8 | 47.9 |
| 10 | 50.9 |
| 10 | 50.9 |
| 15 | 55.2 |
| 20 | 58.4 |
| 25 | 61.0 |
| 50 | 69.4 |
| 75 | 74.3 |
| 100 | 77.8 |
| 125 | 80.4 |
| 150 | 82.4 |
| 175 | 84.0 |
| 200 | 85.3 |
| 225 | 86.3 |
| 250 | 87.2 |
| 275 | 87.9 |
| 300 | 88.6 |
| 325 | 89.1 |
| 350 | 89.5 |
| 375 | 89.8 |
| 400 | 90.1 |
| 425 | 90.4 |
| 450 | 90.5 |
| 475 | 90.7 |
| 500 | 90.8 |

### Edema Reduction in 3 h SCI Contusion Study

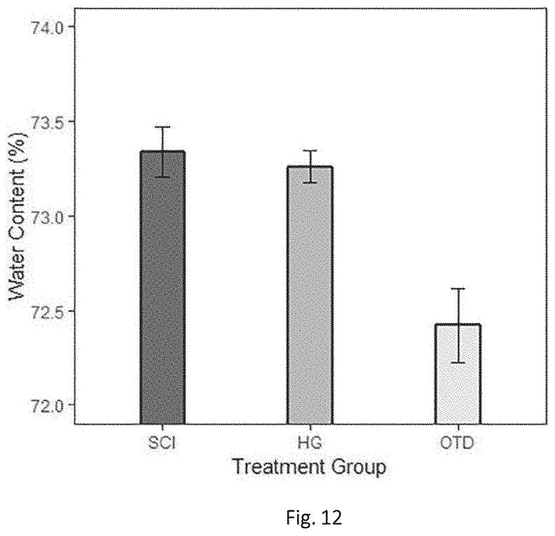

FIG. 12 shows tissue water content (%) for the treatment groups, injured animals receiving no treatment (SCI), injured animals treated with an inoperable OTD with hydrogel (SCI C HG) and injured animals with the operating OTD (SCICOTD). Mean and standard error are shown in Table 11. Injury (SCI, n=5) caused an increase in water content to 73.3±0.30%. The (SCI C HG) case had the entire OTD with hydrogel implanted but did not have flow within the device during the observation period. The results for the (SCI C HG, n=5) case did not significantly differ from the injured, untreated case at 73.3±0.19%. This confirms that the non-operational device had no significant impact, indicating that water content reduction was not due to the hydrogel alone. However, the treatment case with a functional OTD (SCICOTD, n=5) had water content value of 72.4±0.43%. The study results correspond to a reduced tissue water content in OTD treated animals (SCI C OTD) at the lesion epicenter compared to injured, untreated animals (SCI). Water content in the OTD treatment group is remarkably lower than that of SCI group (mean: 73.34% and 95% CI: 73.03-73.65). The treatment effect is −0.92% (95% CI: −1.37 to −0.47%, p<0.0001). However, the treatment effect of HG is not significant (Table 12). For OTD group Cohen's effect size is 0.49, generally seen as a medium level. Although the OTD did not return the tissue to the uninjured water content, it resulted in approximately a 29% reduction in edema compared to the injured group. The significance of this is illustrated in the section “Discussion.”

TABLE 11
Summary on water content in the three treatment groups

| Treatment group | Sample size | Mean (%) | 95% CI | P value |
|---|---|---|---|---|
| HG | 5 | 73.26 | 0.19 | 0.09 |
| OTD | 5 | 72.42 | 0.43 | 0.19 |
| SCI | 5 | 73.34 | 0.30 | 0.13 |

TABLE 12
Treatment effect estimation using a linear regression model

| Treatment group | Group difference in | | |
|---|---|---|---|
| comparison | water content (%) | 95% CI | P value |
| SCI | 73.34 | 73.03-73.65 | <0.0001 |
| OTD vs SCI | −0.92 | −1.37 to 00.47 | <0.0001 |
| HG vs SCI | −0.08 | −0.53-0.37 | 0.57 |

### Discussion

### Edema Progression

In this study, we performed a detailed analysis of the time course of spinal cord water content after severe thoracic contusion SCI in the rat model for the first time. At the lesion epicenter, spinal cord water content was significantly elevated as soon as 1 h after injury, peaked at 72 h at a value of (78.7±0.67)%, and remained elevated at 28 d after injury. At segments 5 mm rostral or caudal to the lesion epicenter, spinal cord water content was elevated 1 d after injury, peaked at 72 h, and returned to baseline by 7 d after injury (see FIG. 3). The total increase in water content during edema progression at the epicenter was 8.5% (up from 70.2% for sham). The sham water content values are consistent with the literature (Sharma, H. S., and Olsson, Y. (1990). Edema formation and cellular alterations following spinal cord injury in the rat and their modification with p-chlorophenylalanine. Acta Neuropathol. 79, 604-610). These data suggest that there is a period of approximately 3 days of peak edema spreading from the injury epicenter radially along the parenchyma of the spinal cord, and thus inform the possible “treatment window” needed for therapeutic spinal cord edema reduction.

### Estimated Water Extraction Rate by the OTD

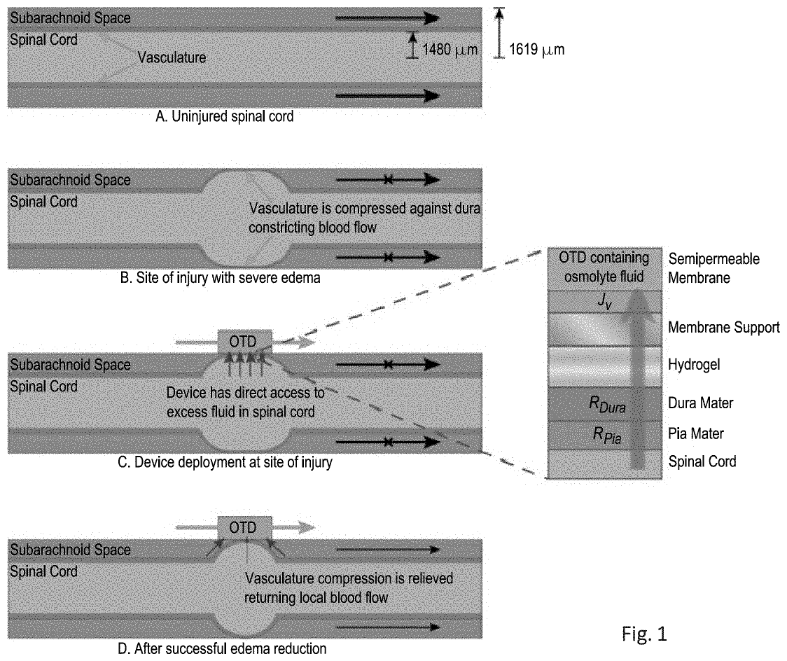

The estimated extraction rate on the order of 30 mL/h for the OTD in the in vivo studies indicates that the device can remove substantially more water than that associated with edema. The estimated geometry implies that the excess water is approximately 7.2 mL of fluid. This is substantially less than the 90 mL of fluid expected to be removed during the 3 h operation of the OTD. It is likely that, during significant swelling, the OTD can extract fluid directly from edema in the cord (FIG. 1). After the swelling radius has reduced to a critical point, extraction of additional fluid is likely from surrounding tissue and the subarachnoid space.

### Relatively Small Increases in % Water Content can Result in Vascular Constriction in the Spinal Cord

Relatively small changes in % water content have been shown to be significant in cerebral edema (Keep, R. F., Hua, Y., and Xi, G. (2012). Brain water content. A misunderstood measurement? Transl. Stroke Res. 3, 263-265; McBride, D. W., Hsu, M. S., Rodgers, V. G. J., and Binder, D. K. (2012). Improved survival following cerebral edema using a novel hollow fiber-hydrogel device. J. Neurosurg. 116, 1389-1394). This is also likely in SCI where constriction in the narrow subarachnoid space can lead to vascular compression. The water content measurement can be used to estimate the degree of radial swelling of the cord at the epicenter that could result in vascular constriction in the subarachnoid space. Using estimates of the spinal cord dimensions and water content results, we developed a first approximation model of spinal cord swelling with respect to water content (illustrated in FIG. 1). The spinal cord is approximated as a uniform cylindrical tube with swelling due toedema represented as a centrally located spherical element. The uninjured volume, Vi, is then

Vᵢ=πLRᵢ² (1)

where L is the length and Ri is the initial radius of the spinal cord. The additional increase in volume, Va, caused by swelling is

$${V}_{a} = \frac{4 π}{3} {( {R}_{s}^{2} - {R}_{i}^{2} )}^{1.5} \qquad ( 2 )$$

where Rs is the swollen radius of the spinal cord. Given the initial and final percent water content and assuming constant density of the fluid associated with the spinal cord, the radius due to swelling can be determined by iteration using the relationship,

$$\frac{\% {water}_{final}}{\% {water}_{initial}} = \frac{4 {( {R}_{s}^{2} - {R}_{i}^{2} )}^{3 / 2}}{3 {R}_{i}^{2} L} + 1 \qquad ( 3 )$$

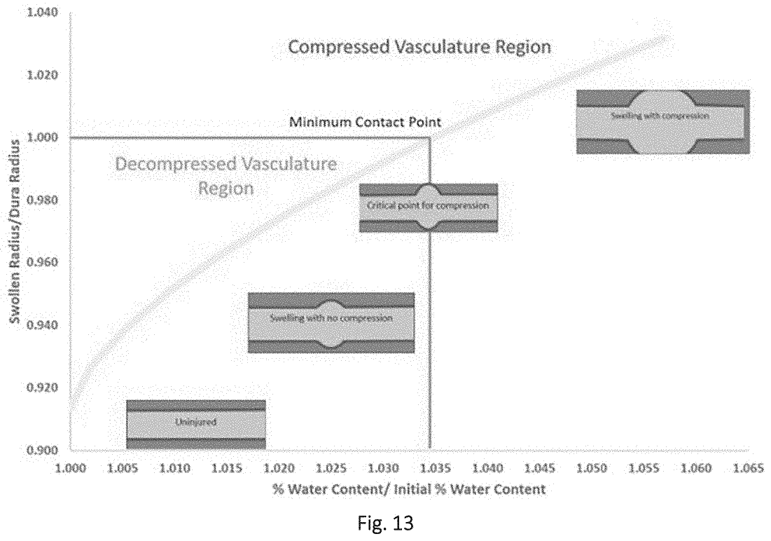

FIG. 13 illustrates the significance of changes in water content to potential vascular constriction for radial swelling at the epicenter for a 5 mm segment of a model rat spinal cord. In this illustration, radial dimensions for the cord (1,480 mm) and subarachnoid space (1,619 mm) are estimated from very high resolution ultrasound images of Wistar rat spinal cord (Soubeyrand, M., Badner, A., Vawda, R., Chung, Y. S., and Fehlings, M. G. (2014a). Very high-resolution ultrasound imaging for real-time quantitative visualization of vascular disruption after spinal cord injury. J. Neurotrauma 31, 1767-1775). Using water content results from this study and the Wistar rat spinal cord dimensions, only a 0.035 decrease in water content ratio (or decrease from 71.8 to 69.4% water content) is required for predicted decompression of the subarachnoid space and hypothetically reduce constriction of the local vasculature. While the spinal cord and subarachnoid space are clearly non-uniform, this estimate addresses how minute increases in % water content due to swelling may lead to constriction of the local vasculature in SCI. This is consistent with experimental results observed by others (Saadoun, S., and Papadopoulos, M. C. (2020). Targeted perfusion therapy in spinal cord trauma. Neurotherapeutics 17, 511-521).

### OTD has Potential Therapeutic Benefits

The comparison between the calculated water content for the threshold for edema water and the value in which the OTD can reduce the water content is remarkably similar, albeit the direct dimensions of the spinal cord tissue used here have not been determined for the Sprague Dawley rats used in this study.

This result implies two important insights: (1) the OTD may reduce swelling to a level of therapeutic significance, and (2) there may be significant therapeutic benefits from reducing the water content by even a relatively small percent. The results from this study shows that the reduction of edema by the OTD (from 73.3±0.30% to 72.4±0.43%) can potentially reducing vascular collapse and opening the subarachnoid space. It is noteworthy, however, that our device functions by removal of water content from the spinal cord through the dura. We anticipate that a severe spinal cord contusion using the IH Impactor (Infinite Horizons impactor, model #IH-0400, Precision Systems and Instrumentation, LLC) may disrupt the collagen and elastin fibers that make up the dura, allowing for water extraction through a disrupted water-tight barrier (Maikos, J. T., Elias, R. A., and Shreiber, D. I. (2008). Mechanical properties of dura mater from the rat brain and spinal cord. J. Neurotrauma 25, 38-51; Soubeyrand, M., Dubory, A., Laemmel, E., Court, C., Vicaut, E., and Duranteau, J. (2014b). Effect of norepinephrine on spinal cord blood flow and parenchymal hemorrhage size in acute-phase experimental spinal cord injury. Eur. Spine J. 23, 658-665). Assessment of dural integrity following contusion injury will be necessary to determine the mechanism of action of our current approach, as well as the long-term viability of above dural treatments. Further, investigation into ISP, SCPP and intraoperative ultrasound imaging to verify vascularity and metabolic state of the tissue following OTD treatment are necessary to further validate our theoretical model and identify the therapeutic potential of this novel approach.

### Scaling to Human Parameters

This analysis can scale to human parameters. We estimate that the OTD can perform well above the therapeutic limit for its application in patients. Assume that swelling volume scales with cord radii and the available surface area of the cord to deploy the OTD increases by an order of magnitude for human. Then assuming the overall hydraulic resistance through the human dura is no more than an order of magnitude of the rat, the effective removal rate for a human would be approximately 90 mL in 3 h, based on our computational studies. In addition, the above studies were performed with the relatively low osmotic pressures, which can be dynamically controlled in the OTD if necessary.

### Methods

### OTD Development

### Design Considerations

The device is primarily structured with a tangential flow module supporting a semipermeable membrane. The membrane is in contact with a hydrogel that rests on the exposed tissue. aCSF containing a rejected osmolyte is passed across the solution side of the membrane. At the membrane surface, the osmolyte in the OTD initiates controlled fluid removal from the tissue where it is expelled with the effluent.

Excess water removal by the OTD requires fluid permeability across the dura mater as well as other tissue between the OTD and the spinal cord core. As shown in FIG. 1, the flux through the OTD must pass in series through the hydratable material and through the semipermeable membranes. Using the Kedem-Katchalsky model for membrane processes, the water flux, Jv, through the device, for a uniform transport area is described as (Kedem, O., and Katchalsky, A. (1958). Thermodynamic analysis of the permeability of biological membranes to non-electrolytes. Biochim. Biophys. Acta 27, 229-246):

$${J}_{v} = \frac{1}{μ} \frac{Δ P - {σ}_{BSA} {Δπ}_{BSA} - {σ}_{other} {Δπ}_{other}}{{R}_{m} + {R}_{membrane support} + {R}_{hydrogel} + {R}_{dura} + {R}_{pia}} \qquad ( 4 )$$

where 1P is the transmembrane pressure driving force, 1p is the osmotic pressure, s is the osmotic reflection coefficient which provides a measure of the membrane permselectivity (approximately unity in our studies), Rₘ is the membrane resistance, R_{membrane} support is the flux resistance due to the membrane support, R_{hydrogel} is the hydratable hydrogel resistance, Rama is the hydraulic resistance due to the dura mater tissue, Rₚᵢₐ is the resistance to the pia mater tissue, and μ is the solution viscosity.

The OTD operates as a standard membrane process except ΔP<Δπ is required to obtain a negative Jv. This is accomplished using low flowrates so that the flux of solvent is into the OTD and away from the tissue. However, in operation, permeating fluid passing through the membrane dilutes the osmolyte at the membrane surface. Since the governing osmotic pressure is associated with the osmolyte concentration immediately at the membrane surface, a computational fluid dynamics model (COMSOL Multiphysics, COMSOL, Inc., Burlington, Mass., United States) was used to estimate the osmotic pressure relative to the internal tangential flow inside the OTD and the resulting permeate flux during operation. Details of the modeling approach are illustrated in the Supplementary Material section “Computational Modeling of Device Efficacy.”

### OTD Operation

The solution chosen was 350 g/L BSA (65,000 MW) solution in 0.15M salt aCSF at pH 7.4. To prepare the solution, aCSF solvent was used to dissolve a weighed amount of BSA (RPI, A30075-100.0X). The solution pH was adjusted using 1 M HCl and 1 M NaOH while undergoing stirring to prevent local denaturation of BSA. The volume of acid and base used to adjust pH was considered part of the solution and was accounted for when determining concentration. The volume of solution considered the specific volume of protein and salt. The computational estimate of the osmotic pressure across the tissue and the membrane was 11.3 kPa.

A Microdyne Nadir, Spectra/Por® 3 10 kDa polyethersulfone (PES) membrane with a support backing of hydrophilic polyethersulfone (PESH) was used for the device membrane. The membrane was chosen for its hydrophilic nature and its rejection of the osmotic agent.

The hydrogel used in this work is 0.3% agar (Sigma, 05040-1KG), by weight, dissolved in aCSF solvent. The agar/aCSF solution was placed in a container to achieve the proper gel height. Next the solution was heated for 30 s in a microwave set to high. Agar was chosen due to its biocompatibility (Tonda-Turo, C., Gnavi, S., Ruini, F., Gambarotta, G., Gioffredi, E., Chiono, V., et al. (2017). Development and characterization of novel agar and gelatin injectable hydrogel as filler for peripheral nerve guidance channels. J. Tissue Eng. Regen. Med. 11, 197-208). Although the water content is higher than that of the tissue, the watery consistency of the gel insures that the device maintains contact with the tissue.

### Selected Operating Conditions

The process was operated with a fixed head pressure of 2.9 kPa to ensure a negative flux (FIG. 4, D). This resulted in an operating flowrate tangential to the membrane of 25 mL/min. We determined the initial osmotic pressure of 350 g/L BSA in aCSF to be 131.4 kPa. The estimated osmotic pressure during operation was reduced to approximately 8 kPa. The conservative estimated overall permeate flux across the membrane was determined to be on the order of 30 mL/h. We recently demonstrated with densimetry methods that the computational analysis was consistent with experimental observation (Hale, C. S., Bhakta, H. C., Jonak, C. R., Yonan, J. M., Binder, D. K., Grover, W. H., et al. (2019). Differential densimetry: a method for determining ultra-low fluid flux and tissue permeability. AIP Adv. 9:095063). These results were determined by CFD model calculations and a 50% reduction in flux due to the expected resistance from the dura and pia tissue (see Table 13).

TABLE 13
Simulated Extraction Rate Dependence on Maximum
Mesh Size for Membrane Device with an Inlet Flowrate of
25 μL min⁻¹ of Bovine Serum Albumin in Artificial
Cerebral Spinal Fluid at pH 7.4, 25° C.

| Maximum | |
|---|---|
| Mesh Size | Extraction Rate |
| (μm) | (μL h⁻¹) |
| 10 | 69.82 |
| 9 | 70.81 |
| 8 | 70.73 |
| 7 | 69.81 |
| 6 | 64.11 |
| 5 | 63.77 |
| 4 | 64.17 |
| 3 | 61.84 |
| 2 | 61.28 |
| 1 | 60.99 |
| 0.9 | 60.99 |
| 0.8 | 60.99 |
| 0.7 | 60.99 |
| 0.6 | 61.00 |
| 0.5 | 61.01 |
| 0.4 | 61.04 |
| 0.3 | 61.08 |

Mesh size of 1 μm was used in this study. Extraction rates for actual transport through tissue are reduced by a factor of two to project additional permeate resistances. Thus, the conservative estimate of 30 μL/h is used in this study.

Osmotic Pressure Data are shown in Table 14 for Bovine Serum Albumin in Artificial Cerebral Spinal Fluid at pH 7.4, 25° C.

TABLE 14

| [BSA] | Osmotic |
|---|---|
| (gL⁻¹ SoIn) | Pressure (kPa) |
| 290 | 99.9 |
| 298 | 77.5 |
| 306 | 87.8 |
| 341 | 134.1 |
| 359 | 135.4 |
| 361 | 130.2 |
| 362 | 143.1 |
| 378 | 176.0 |
| 396 | 196.9 |
| 399 | 246.2 |
| 408 | 141.4 |
| 414 | 243.4 |
| 416 | 264.0 |
| 436 | 390.1 |

### Spinal Cord Injury

Rats were anesthetized with isoflurane inhalation and given an intraperitoneal injection of ketamine and xylazine (K/X) (80/10 mg/kg). We evolved toward isoflurane induction then used ketamine/xylazine injection anesthesia to avoid hemorrhage. With this regimen, we were able to get (1) a reproducible and titratable level of anesthesia appropriate for these experiments; (2) lack of motion of the spine/spinal cord during device application; and (3) lack of hemorrhage. This method also insured that any effect on hemodynamics would be similar across mice given the same anesthetic regimen.

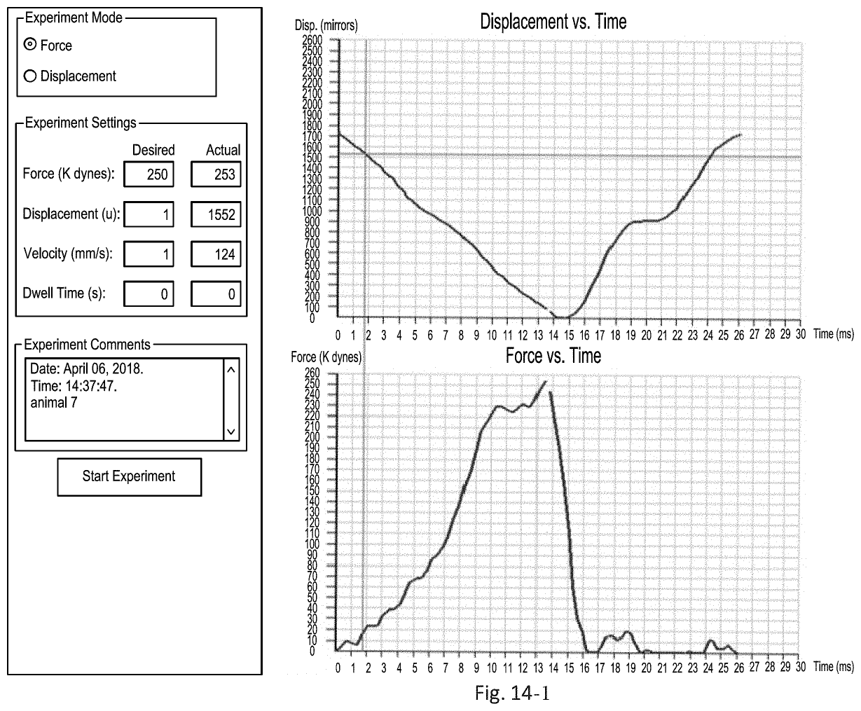

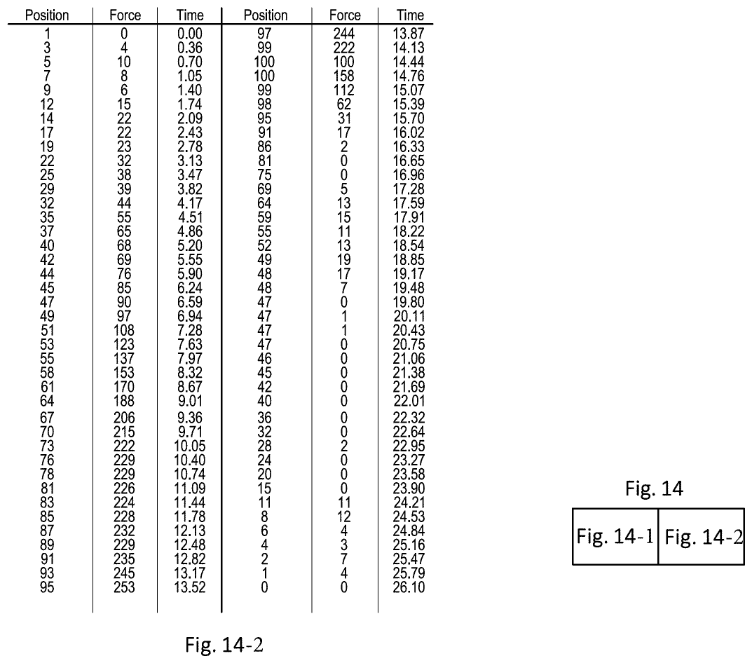

Rats were aseptically prepared for surgery and artificial tear ointment was applied to the eyes to prevent drying. Toe pinch reflex was used to measure anesthetic depth every 10 min throughout the surgery, and supplemental doses of K/X were administered, as needed. A midline incision 2-3 cm long was made along the dorsal surface of the animal and overlying muscle was separated to allow visualization of the spinal column. A laminectomy was performed at thoracic level 8 (T8). For the injury groups, the Infinite Horizons (IH) impactor (Infinite Horizons impactor, model #IH-0400, Precision Systems and Instrumentation, LLC) was used to produce a severe contusion injury of the spinal cord. The exposed cord was contused with a 250 kilodyne (kD) force using a 2.5 mm probe centered along the dorsal column using standard methods (Scheff, S. W., Rabchevsky, A. G., Fugaccia, I., Main, J. A., and Lumpp, J. E. (2003). Experimental modeling of spinal cord injury: characterization of a force-defined injury device. J. Neurotrauma 20, 179-193; Moreno-Manzano, V., Rodriguez-Jimenez, F. J., Garcia-Rosello, M., Lainez, S., Erceg, S., Calvo, M. T., et al. (2009). Activated spinal cord ependymal stem cells rescue neurological function. Stem Cells 27, 733-743; Beggs, L. A., Ye, F., Ghosh, P., Beck, D. T., Conover, C. F., Balaez, A., et al. (2015). Sclerostin inhibition prevents spinal cord injury-induced cancellous bone loss. J. Bone Miner. Res. 30, 681-689). Example impact statistics are shown in the FIG. 14. Control animals received a laminectomy only. Following impact, the cord was examined for adequate bilateral bruising, overlying vertebral muscles were closed with 5-0 chromic gut sutures and skin was closed with 9 mm wound clips.

### Device Mounting

For animals receiving OTD placement, spinal cord exposure and injuries were produced as previously described. Following laminectomy and/or contusion injury, the dorsal processes of the T7 and T9 lamina were removed and flattened to accommodate the length of the device and allow direct contact between the OTD and the underlying tissue at T8 (FIGS. 4, B and C). Following device placement, the overlying vertebral muscles were closed with 5-0 chromic gut sutures and skin was closed with wound clips.

### Post-Operative Care

Post-operative care was performed on animals included in the edema time course. Post-operatively, rats were placed on alpha-dri bedding on a 37° C. water jacket to maintain adequate body temperature. Rats were monitored daily for general health, mobility in the cage, adequate feeding, proper hydration, and signs of distress, including weight loss, piloerection, and porphyrin. Animals were given lactated ringers (5 ml/100 g) for hydration and baytril (5 mg/kg) to prevent infection for 7 days following injury. Animals received buprenorphine (0.5 mg/kg) immediately after surgery and 4 h post-surgery. Buprenorphine administration was continued two times per day (every 12 h) for another 3 days post-surgery. Finally, animals underwent manual bladder expression until bladder function was recovered (typically within 1-2 weeks post injury).

### Water Content

At each experimental endpoint, animals were sacrificed with Fatal Plus (100 mg/kg given I.P.) followed by cardiac puncture, after which 5 mm of spinal cord centered at the injury epicenter, as well as rostral and caudal to the injury (15 mm total), were rapidly dissected and assessed for spinal cord water content. Freshly dissected tissue was placed on a pre-weighted piece of foil and the tissue weight was recorded. Tissue was then dried in an oven at 85° C. for 48 h and reweighed. Percent water content was calculated as (wet weight−dry weight)/wet weight×100. This method allowed for a measure of edema within and immediately surrounding the lesion site.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference in their entireties.

Some embodiments have been described in connection with the accompanying drawing. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed considering the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the embodiments disclosed in the present disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

It is contemplated that various combinations or sub-combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% includes 96%, 97%, 98%, 99%, and 100% as compared to a reference.

Any titles or subheadings used herein are for organization purposes and should not be used to limit the scope of embodiments disclosed herein.

What is claimed is:

1. A continuous-flow system for the treatment of edema in an injured central nervous system (CNS) tissue, comprising:
(a) a reversibly implantable device comprising:
(i) an inflow pathway comprising a first inlet and a first outlet,
(ii) an outflow pathway comprising a second inlet and a second outlet, and
(iii) a fluid flow pathway connecting the first outlet of the inflow pathway and the second inlet of the outflow pathway, wherein the fluid flow pathway comprises a semi-permeable membrane,
(b) a first reservoir;
(c) a fluid-driving apparatus;
(d) a second reservoir; and
(e) a plurality of fluid flow conduits that fluidically connect the first reservoir, the fluid-driving apparatus, the second reservoir, and the reversibly implantable device;
wherein the reversibly implantable device is configured to allow direct contact between the semi-permeable membrane and at least a portion of the injured CNS tissue;
wherein the first reservoir is configured to contain a solution;
wherein the fluid-driving apparatus is configured to pump the solution from the first reservoir, through a conduit, and to the second reservoir;
wherein the second reservoir comprises a vessel and an overflow conduit, such that a head pressure is maintained in the continuous-flow system;
wherein the second reservoir comprises an outlet that is fluidically coupled to the inlet of the inflow pathway of the reversibly implantable device via a fluid flow conduit; and
wherein the solution can pass through the fluid flow pathway, induce osmotic flow of water from the injured CNS tissue across the semipermeable membrane and into the solution, and deliver the water back to the first reservoir.

2. The continuous flow system according to claim 1, wherein the solution comprises a solute selected from the group of a protein, a carbohydrate, a polysaccharide and a polymer.

3. The continuous flow system of claim 1, wherein the semipermeable membrane comprises a material selected from the group consisting of polynephron, polyflux, polysulfone and regenerated cellulose.

4. The continuous-flow system of claim 1, wherein the semipermeable membrane has a molecular weight cut-off of between about 1 to 60 kilodaltons (kDa).

5. The continuous-flow system of claim 1, wherein an outer diameter of the fluid flow pathway is 1-2 cm and an inner diameter of the fluid flow pathway is 0.5-1.6 cm.

6. The continuous flow system of claim 1, wherein one or more of the inflow pathway comprising a first inlet and a first outlet; the outflow pathway comprising a second inlet and a second outlet and the fluid flow pathway connecting the first outlet of the inflow pathway and the second inlet of the outflow pathway are removably connected to the continuous-flow system.

7. The continuous-flow system according to claim 1, wherein the fluid flow path of the reversibly implantable device, including the semipermeable membrane, conforms to the surface of the traumatically injured CNS tissue.

8. The continuous-flow system according to claim 1, wherein osmotic pressure of the solution is controlled in real time by temperature and/or solute concentration in response to feedback monitoring of a degree of swelling of the CNS tissue, and wherein the system operates on a time scale on the order of a swelling rate to stabilize the tissue.

9. The continuous flow system according to claim 1, wherein the fluid-driving apparatus is a pump or a gravity feed system.

10. A method for removing water from a traumatically injured central nervous system (CNS) tissue in a subject in a controlled fashion, the method comprising:
(a) exposing a surface of the traumatically injured CNS tissue;
(b) applying a hydrogel to the exposed surface of the traumatically injured CNS tissue, wherein the hydrogel is permeable and allows passage of water,
(c) placing the semipermeable membrane of the reversibly implantable device of the continuous-flow system of claim 1 in contact with the hydrogel; and
(d) flowing or pumping through a lumen of the fluid flow pathway a concentrated solution of a solute that produces a concentration-dependent osmotic pressure, wherein the solute cannot pass through the semi-permeable membrane, wherein the concentrated solution of the solute in the fluid flow pathway of the reversibly implantable device induces an osmotic pressure that draws water from the tissue into the hydrogel and then into the semipermeable membrane, where the water is removed and carried away from the hydrogel and the tissue.

11. The method according to claim 10, wherein the solute is a globular protein.

12. The method of claim 11, wherein the globular protein is bovine serum albumin (BSA).

13. The method according to claim 12, wherein the BSA is at a concentration of about 350 g/L.

14. The method according to claim 10, wherein the CNS tissue is a spinal tissue.

15. The method according to claim 10, wherein the fluid flow path of the reversibly implantable device, including the semipermeable membrane, conforms to the surface of the traumatically injured CNS tissue.

16. The method according to claim 10, wherein the reversibly implantable device is attached to the subject with an adhesive.

17. The method according to claim 10, wherein a concentration of the solute that cannot pass through the semi-permeable membrane is changed or modified over time to alter the rate of water removal.

18. The method according to claim 17, wherein a concentration of the globular protein that cannot pass through the semi-permeable membrane is altered to between about 0.1 to about 50% to alter the rate of water removal.

19. The method of claim 10, wherein the pressure of the solution passed across the semi-permeable membrane is altered to change or modify the rate of water removal.

20. The method according to claim 10, wherein the temperature of the concentrated solution is changed in the range of about 20° C. to about 40° C. to alter the rate of water removal.

21. The method according to claim 10, wherein the injured central nervous system (CNS) tissue is associated with the spinal column and the surface of the traumatically injured CNS tissue is exposed by removing or folding back of dorsal processes of vertebra or vertebrae.

22. The method according to claim 10, wherein the hydrogel has a sufficient permeability to allow passage of nutrients, drugs, ions, and water, and wherein the concentrated solution of a solute contains a nutrient, drug or ion.
